(19) United States
(12) Patent Application Publication
(10) Pub. No.: US 2011/0009469 A1
(43) Pub. Date: Jan. 13, 2011

(54) COMPOSITIONS AND METHODS OF TREATING NEOPLASIA

(75) Inventors: Joshua T. Mendell, Baltimore, MD (US); Oliver Andrew Kent, Baltimore, MD (US); Anirban Maitra, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/746,517

(22) PCT Filed: Dec. 5, 2008
(86) PCT No.: PCT/US08/13415

§ 371 Date: Sep. 23, 2010

Related U.S. Application Data
(60) Provisional application No. 61/005,588, filed on Dec. 5, 2007.

(51) Int. Cl. A61K 31/7105; A61K 31/351; A61K 31/135; A61K 31/277; C12N 15/63; G01N 33/53; C40B 40/06; C12N 5/09; C12Q 1/68; A61P 35/00

(52) U.S. Cl. 514/44 A; 514/456; 514/646; 514/523; 435/320.1; 436/94; 506/16; 435/375; 435/6

(57) ABSTRACT

The present invention provides compositions and methods featuring microRNA polynucleotides for the diagnosis, treatment or prevention of neoplasia.

### CROSS-REFERENCE TO RELATED APPLICATION

This application claims the benefit of the following U.S. Provisional Application No. 61/005,588, filed on Dec. 5, 2007, the entire contents of which are incorporated herein by reference.

### BACKGROUND OF THE INVENTION

MicroRNAs (miRNAs) are approximately 21-24 nucleotide RNA molecules that regulate the translational efficiency and stability of target messenger RNAs (mRNAs). In humans, nearly 500 miRNAs have been identified that are predicted to regulate at least one third of all mRNA transcripts. Numerous studies have documented that dysregulated miRNA expression is a very frequent, if not ubiquitous, feature of human cancers. Abnormal miRNA expression profiles have been described in many diverse tumor types. miRNA expression signatures are not only highly characteristic of specific cancer subtypes and therefore useful for tumor classification, but also have been associated with prognosis, staging, and response to therapy. Moreover, a causative role for altered miRNA expression in cancer is suggested by the demonstration that select miRNAs exhibit oncogenic and tumor suppressor activity.

Cancer causes one in every four US deaths and is the second leading cause of death among Americans. Pancreatic ductal adenocarcinoma (PDAC) is one of the most lethal human malignancies. PDAC carries an extremely poor prognosis, typically presenting with metastasis at the time of diagnosis and exhibiting resistance to conventional therapies. In most patients, pancreatic cancer is advanced by the time the disease is diagnosed, and the disease has advanced to the stage surgery is no longer useful. Typically, patients with PDAC are given radiotherapy and/or chemotherapy, but these treatments are rarely effective. The vast majority of patients diagnosed with PDAC die within a year of diagnosis. Improved compositions and methods for the diagnosis, treatment or prevention of neoplasia, particularly of pancreatic neoplasias are urgently required.

### SUMMARY OF THE INVENTION

As described below, the present invention features compositions and methods for the diagnosis, treatment or prevention of neoplasia.

In one aspect, the invention generally provides a method of preventing or reducing tumorogenesis in a subject (e.g., human patient), the method involving administering to the subject an agent that increases miR-143, miR-145, and/or miR-27b expression relative to a reference cell, thereby reducing or preventing tumor formation.

In another aspect, the invention provides a method of treating or preventing a neoplasia in a subject, the method involving administering to the subject an agent that increases miR-143, miR-145, and/or miR-27b expression, thereby treating or preventing the neoplasia.

In a related aspect, the invention provides a method of increasing miR-143, miR-145, and/or miR-27b expression in a neoplastic cell, the method involving contacting the cell with an agent that increases miR-143, miR-145, and/or miR-27b expression.

In another aspect, the invention provides a method of treating or preventing a neoplasia, the method involving contacting a neoplastic cell having increased Kras signaling with an agent that inhibits a MAPK signaling pathway component or increases miR-143, miR-145, and/or miR-27b expression, thereby treating or preventing the neoplasia.

In yet another aspect, the invention provides a expression vector containing a polynucleotide encoding miR-143, miR-145, and/or miR-27b positioned for expression in a mammalian cell. In one embodiment, the vector is a viral expression vector (e.g., an adenoviral vector, an adeno-associated viral vector, or a lentiviral vector).

The invention further provides a cell (e.g., a bacterial cell or a mammalian cell) containing the vector of the previous aspect.

In another aspect, the invention provides a pharmaceutical composition for the treatment of neoplasia involving an effective amount of an isolated miR-143, miR-145, and/or miR-27b polynucleotide in a pharmaceutically acceptable excipient. In one embodiment, the polynucleotide is a microRNA.

In another aspect, the invention provides a pharmaceutical composition for the treatment of neoplasia containing an effective amount of a vector encoding a miR-143, miR-145, and/or miR-27b microRNA in a pharmaceutically acceptable excipient.

In various embodiments of the previous aspect, the composition is labeled for the treatment of a pancreatic cancer.

In another aspect, the invention provides a kit for the treatment of a neoplasia, the kit containing an effective amount of an agent that increases miR-143, miR-145, and/or miR-27b expression, and written instructions for using the kit.

In another aspect, the invention provides a method for identifying or characterizing a neoplasia in a subject, the method involving detecting miR-143, miR-145, miR-27b, miR-31, miR-34a and miR-199b in a biological sample derived from the subject, thereby identifying or characterizing the neoplasia.

In yet another aspect, the invention provides a method for diagnosing a subject as having or having a propensity to develop a neoplasia, the method involving measuring the level of a marker selected from the group consisting of miR-143, miR-145, miR-27b, miR-31, miR-34a and miR-199b in a biological sample from the subject, and detecting an alteration in the level of the marker in the sample relative to the level in a control sample, wherein detection of an alteration in the marker level indicates the subject has or has a propensity to develop a neoplasia.

In embodiments of the previous aspects, the method identifies a reduction in miR-143, miR-145, miR-27b level or an increase in miR-31, miR-34a and miR-199b level.

In yet another aspect, the invention provides a method for identifying the prognosis of a subject having a neoplasia, the method involving detecting the level of miR-143, miR-145, miR-27b level, miR-31, miR-34a and/or miR-199b in a subject, wherein an decrease (e.g., 5%, 10%, 15%, 25% or more) in the level of miR-143, miR-145, miR-27b level or an increase in the level of miR-31 and miR-199b identifies the subject as having a poor prognosis. In one embodiment, the subject having a poor prognosis is identified as in need of aggressive therapy.

In another aspect, the invention provides a method for selecting a therapy for a subject having a neoplasia, the method involving detecting Kras or MAPK signaling in a biological sample derived from the subject, wherein the level of Kras signaling or a increase in MAPK signaling relative to a reference is indicative of the efficacy of said therapy.

In another aspect, the invention provides a method for selecting a therapy for a subject having a neoplasia, the method involving detecting miR-143, miR-145, miR-27b, miR-31, miR-34a and/or miR-199b in a biological sample derived from the subject, wherein the level of miR-143, miR-145, miR-27b, miR-31 and/or miR-199b relative to a reference is indicative of the efficacy of said therapy.

In another aspect, the invention provides a microarray containing at least two polynucleotides selected from the group consisting of miR-143, miR-145, miR-27b, miR-31, miR-34a and miR-199b. In one embodiment, the microarray comprises at least miR-143, miR-145, and miR-27b. In another embodiment, the microarray further comprises miR-31, miR-34a and miR-199b.

In another aspect, the invention provides a nucleic acid probe that hybridizes with a microRNA sequence selected from the group consisting of miR-143, miR-145, miR-27b, miR-31, miR-34a and miR-199b.

In another aspect, the invention provides a method of preventing or reducing tumor formation, the method involving contacting a neoplastic cell with an agent that reduces miR-31, miR-34a and/or miR-199b expression or biological activity, thereby reducing or preventing tumor formation.

In another aspect, the invention provides a method of treating or preventing a neoplasia in a subject, the method involving administering to the subject an agent that reduces miR-31, miR-34a and/or miR-199b expression, thereby treating or preventing the neoplasia.

In another aspect, the invention provides a vector containing a polynucleotide encoding a miR-31, miR-34a and miR-199b inhibitory nucleic acid molecule. In one embodiment, the inhibitory nucleic acid molecule is an antisense, siRNA, or shRNA molecule.

In another aspect, the invention provides a pharmaceutical composition for the treatment of a neoplasia, the composition containing an effective amount of a miR-31, miR-34a and miR-199b inhibitory nucleic acid molecule. In one embodiment, the inhibitory nucleic acid molecule is an antisense, siRNA, or shRNA molecule.

In another aspect, the invention provides a method for increasing miR143 and/or miR145 expression in a cell, the method involving contacting the cell with an agent that inhibits RREB-1, thereby increasing the expression of miR-143 and/or 145 primary transcript or mature miRNA.

In another aspect, the invention provides a method for increasing miR143 and/or miR145 expression in a cell, the method involving contacting the cell with an agent that inhibits a component of a MAPK pathway, thereby increasing the expression of miR-143 and/or 145. In one embodiment, the cell has increased Kras signaling.

In another aspect, the invention provides a method of identifying an agent that treats or prevents a neoplasm, the method involving contacting a cell that expresses a microRNA selected from the group consisting of miR-143, miR-145, miR-27b, miR-31, miR-34a and miR-199b with an agent, and comparing the level of microRNA expression in the cell contacted by the agent with the level of expression in a control cell, wherein an agent that alters microRNA expression thereby treats or prevents a neoplasm.

In various embodiments of the above aspects or of any method delineated herein, the alteration (increase or decrease) is a reduction in miR-31, miR-34a and miR-199b or an increase in miR-143, miR-145, miR-27b. For example, an alteration in expression by at least about 5%, 10%, 20%, 25%, 30%, 50%, 75%, 85%, 95% or more relative to a reference (e.g., an untreated control cell). In other embodiments of the above aspects, the neoplastic cell has increased Kras signaling relative to a reference. In still other embodiments, the neoplasia is a pancreatic cancer. In other embodiments, the untreated neoplastic cell is characterized as having a reduced level of miR-143, miR-145, and/or miR-27b. In various embodiments of the above aspects or of any method delineated herein, the agent inhibits the expression or biological activity of a component of a MAPK signaling pathway component. Agents for use in a method delineasted herein include PD98059 [2′-amino-3′-methoxyflavone](Calbiochem Corp., La Jolla, Calif.) and PD184352 (CI-1040) [2-(2-chloro-4-iodo-phenylamino)-Ncyclopropylmethoxy-3,4-difluoro-benzamide] and U0126 [1,4-diamino-2,3-dicyano-1,4-bis(2-aminophenylthio)butadiene].

In other embodiments of methods delineated herein, an agent of the invention is an expression vector containing a polynucleotide encoding miR-143, miR-145, and/or miR-27b. In other embodiments, detection of an increase in Kras signaling or a increase in MAPK signaling relative to a reference indicates that the subject is likely to benefit from an increase in miR-143, miR-145, miR-27b expression or biological activity. In other embodiments of methods delineated herein, detection of an increase in Kras signaling or a increase in MAPK signaling relative to a reference indicates that the subject is likely to benefit from a reduction in miR-31, miR-34a and/or miR-199b expression. In other embodiments of methods delineated herein, detection of a decrease in miR-143, miR-145, miR-27b or an increase in miR-31, miR-34a and/or miR-199b relative to a reference indicates that the subject is likely to benefit from aggressive anti-neoplasia therapy. In other embodiments, the neoplasia has increased Kras signaling relative to a reference. In other embodiments of methods delineated herein, an agent of the invention is a miR-31, miR-34a or miR-199b inhibitory nucleic acid molecule (e.g., an antisense, siRNA, or shRNA molecule). In other embodiments, the agent is an expression vector containing a polynucleotide encoding a miR-31, miR-34a and miR-199b inhibitory nucleic acid molecule. In various embodiments of a method delineated herein, detection is by Rt-PCR, hybridization, Northern blot, quantitative PCR, or ribnucleaase protection assay.

The invention provides compositions and methods featuring microRNAs for the diagnosis, treatment or prevention of neoplasia (e.g., pancreatic cancer). Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Kras” is meant a polypeptide having at least about 85% identity to the amino acid sequence provided at Genbank Accession No. AAB41942 or ABY87538. Kras and its association with cancer is described by Kranenburg, “The KRAS oncogene: past, present, and future,” Biochim. Biophys. Acta 1756: 81-82, 2005 and by Lee et al., “Clinicopathologic significance of the K-ras gene codon 12 point mutation in stomach cancer: an analysis of 140 cases,” Cancer 75: 2794-2801, 1995, each of which is incorporate herein by reference in its entirety.

Two exemplary amino acid sequences of Kras are provided below.

| | |
|---|---|
| 1 | mteyklvvvg acgvgksalt iqliqnhfvd eydptiedsy rkqvvidget clldildtag |
| 61 | qeeysamrdq ymrtgegflc vfainntksf edihhyreqi krvkdsedvp mvlvgnkcdl |
| 121 | psrtvdtkqa qdlarsygip fietsaktrq gvddafytiv reirkhkekm skdgkkkkkk |
| 181 | sktkcvim |
| 1 | mteyklvvvg aggvgksalt iqliqnhfvd eydptiedsy rkqvvidget clldildtag |
| 61 | qeeysamrdq ymrtgegflc vfainntksf edihhyreqi krvkdsedvp mvlvgnkcdl |
| 121 | psrtvdtkqa qdlarsygip fietsaktrq rvedafytiv reirqyrlkk iskeektpgc |
| 181 | vkikkciim. |

By “Kras signaling” is meant Kras GTPase activity, GTP/GDP binding activity or any other signal transduction activity.

By “MAPK signaling” is meant Map kinase activity, or any downstream signal transduction activity within the MAPK pathway.

By “MAPK signaling pathway component” is meant any polypeptide that function in mitogen activated protein kinase signal transduction. Exemplary MAPK signaling pathway components include, but are not limited to, RAF, MEK, and MAPK.

By “miR-34a” is meant a microRNA comprising or having at least 85% identity to the mature microRNA nucleic acid sequence provided at Genbank Accession No. EF592573 and having miR-34a biological activity.

An exemplary mir-34a primary transcript sequence is provided below:

| | |
|---|---|
| 1 | gacccctccc ccgcaccgac gtgattcgga tcgcgcggtg ctggcgccgc cttcatgcgc |
| 61 | cctgcctggc ccccacctgg tcctctttcc ttttcaggtg gaggagatgc cgctgtcccg |
| 121 | tcggtctggg gacagcccag ctccccggat cccgggctgg agagacgcgt cgcggccccg |
| 181 | gggcctggtg gcacngagca ggaaggagga cccggcggcg ggctctgcct gggcttgcct |
| 241 | gggcttgttc cgagccgggc tgcttctcgg tgaccacgca gatcgggggc atttggagat |
| 301 | tttgcgggag tcctgcagcc aagctccggg gcaggagagg cctggaagcc tgcactacct |
| 361 | gctcgccccg tcccagcatg cacccaggtg ctggggagag gcaggacagg cctgtccccc |
| 421 | gagtcccctc cggatgccgt ggaccggcca gctgtgagtg tttctttggc agtgtcttag |
| 481 | ctggttgttg tgagcaatag taaggaagca atcagcaagt atactgccct agaagtgctg |
| 541 | cacgttgtgg ggcccaagag ggaagatgaa gcgagagatg cccagaccag tgggagacgc |
| 601 | caggacttcg gaagctcttc tgcgccacgg tgggtggtga gggcggctgg gaaagtgagc |
| 661 | tccagggccc caggagcagc ctgctcgtgg gtgcggaagg aaaaaggcac aggggcttgg |
| 721 | tgtgggcggc ttttggctgg gagaagtttg cacgtaggga gaatagtagc cagtgtttgc |
| 781 | agagcactta ctatgcagga aggcctgtcc taagtattgt aagtgtatta catcatgtac |
| 841 | aagtgtctgt gattaacccc gtcttgcaga gaaggaaaca aaagtacaaa cagaaaatgt |
| 901 | aactaagcat gcaattaata aaaagggacc aggttttgaa cgcga |

An exemplary mature miR-34a sequence: uggcagugucuuagcugguugu.

By “miR-199b” is meant a microRNA comprising or having at least 85% identity to the nucleic acid sequence provided at Genbank Accession No. AJ550412 and having miR-199b biological activity.

An exemplary miR-199b nucleic acid sequence follows: 1 cccagtgttt agactatctg ttc.

By “miR-31” is meant a microRNA comprising or having at least 85% identity to the nucleic acid sequence provided at Genbank Accession No. AJ421753 and having miR-31 biological activity.

An exemplary miR-31 nucleic acid sequence follows: 1 ggcaagatgc tggcatagct g

By “miR-27b” is meant a microRNA comprising or having at least 85% identity to the nucleic acid sequence provided at Genbank Accession No. AJ459719 and having miR-27b biological activity. Genbank Accession No. AJ459719 refers to the mouse sequence, which is identical to the human sequence.

An exemplary miR-27b nucleic acid sequence follows: uucacaguggcuaaguucugc

By “miR-143” is meant a microRNA comprising or having at least 85% identity to the nucleic acid sequence provided at Genbank Accession No. AJ535834 and having miR-143 biological activity.

An exemplary miR-143 nucleic acid sequence follows: 1 tgagatgaag cactgtagct c

By “miR-145” is meant a microRNA comprising or having at least 85% identity to the nucleic acid sequence provided at Genbank Accession No. AJ535835 and having miR-145 biological activity.

An exemplary mir-145 nucleic acid sequence follows: 1 gtccagtttt cccaggaatc c

By “miR-34a, mir-199b, miR-31, miR-27b, miR-143, or miR-145 biological activity” is meant a microRNA having messenger RNA regulatory activity.

By “tumorogenesis” is meant the development, production, or formation of a tumor.

By “agent” is meant a polypeptide, polynucleotide, or fragment, or analog thereof, small molecule, or other biologically active molecule.

By “alteration” is meant a change (increase or decrease) in the expression levels of a polynucleotide or polypeptide as detected by standard art known methods such as those described above. As used herein, an alteration includes a 5% or 10% change in expression levels, preferably a 25% change, more preferably a 40% change, and most preferably a 50% or greater change in expression levels.

By “antisense molecule” is meant a non-enzymatic nucleic acid molecule or analog or variant thereof that binds to a target nucleic acid molecule sequence by means of complementary base pairing, such as an RNA-RNA or RNA-DNA interactions and alters the expression of the target sequence. Typically, antisense molecules are complementary to a target sequence along a single contiguous sequence of the antisense molecule. In certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of a target sequence.

The phrase “in combination with” is intended to refer to all forms of administration that provide a first agent together with a second agent, such as a second inhibitory nucleic acid molecule or a chemotherapeutic agent, where the two are administered concurrently or sequentially in any order.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an inhibitory nucleic acid molecule is in large part complementary to a target sequence. The complementarity need not be perfect, but may include mismatches at 1, 2, 3, or more nucleotides.

By “control” is meant a standard or reference condition.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more.

“Detect” refers to identifying the presence, absence or amount of the object to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include bacterial invasion or colonization of a host cell.

By “an effective amount” is meant the amount of an agent required to ameliorate the symptoms of a disease relative to an untreated patient. The effective amount of active agent(s) used to practice the present invention for therapeutic treatment of a neoplasia varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “increases” is meant an increase by at least about 5% relative to a reference level. An increase may be by 5%, 10%, 15%, 20%, 25% or 50%, or even by as much as 75%, 85%, 95% or more.

By “inhibits a neoplasia” is meant decreases the propensity of a cell to develop into a neoplasia or slows, decreases, or stabilizes the growth, proliferation, or metastasis of a neoplasia.

By “inhibitory nucleic acid molecule” is meant a single stranded or double-stranded RNA, siRNA (short interfering RNA), shRNA (short hairpin RNA), or antisense RNA, or a portion thereof, or an analog or mimetic thereof, that when administered to a mammalian cell results in a decrease (e.g., by 10%, 25%, 50%, 75%, or even 90-100%) in the expression of a target sequence. Typically, a nucleic acid inhibitor comprises or corresponds to at least a portion of a target nucleic acid molecule, or an ortholog thereof, or comprises at least a portion of the complementary strand of a target nucleic acid molecule.

By “marker” is meant any protein or polynucleotide having an alteration in expression level or activity that is associated with a disease or disorder.

The term “microarray” is meant to include a collection of nucleic acid molecules or polypeptides from one or more organisms arranged on a solid support (for example, a chip, plate, or bead).

By “modification” is meant any biochemical or other synthetic alteration of a nucleotide, amino acid, or other agent relative to a naturally occurring reference agent.

By “neoplasia” is meant any disease that is caused by or results in inappropriately high levels of cell division, inappropriately low levels of apoptosis, or both. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, pancreatic ductal adenocarcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant microRNA molecule described herein).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“Probe set” or “Primer set” means a set of oligonucleotides that may be used in any biochemical assay or procedure. Exemplary uses for probes/primers include detection of a target nucleic acid or in PCR.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence.

By “reporter gene” is meant a gene encoding a polypeptide whose expression may be assayed; such polypeptides include, without limitation, green fluorescent protein (GFP), glucuronidase (GUS), luciferase, chloramphenicol transacetylase (CAT), and beta-galactosidase.

By “siRNA” is meant a double stranded RNA. Optimally, an siRNA is 18, 19, 20, 21, 22, 23 or 24 nucleotides in length and has a 2 base overhang at its 3′ end. An siRNA is a double stranded RNA that “corresponds” to or matches a reference or target gene sequence. This matching need not be perfect so long as each strand of the siRNA is capable of binding to at least a portion of the target sequence. SiRNA can be used to inhibit gene expression, see for example Bass, 2001, Nature, 411, 428 429; Elbashir et al., 2001, Nature, 411, 494 498; and Zamore et al., Cell 101:25-33 (2000).

dsRNAs can be introduced to an individual cell or to a whole animal; for example, they may be introduced systemically via the bloodstream. Such siRNAs are used to down-regulate mRNA levels or promoter activity.

The term “subject” is intended to include vertebrates, preferably a mammal. Mammals include, but are not limited to, humans.

The term “pharmaceutically-acceptable excipient” as used herein means one or more compatible solid or liquid filler, diluents or encapsulating substances that are suitable for administration into a human.

By “specifically binds” is meant a molecule that recognizes and binds a protein or nucleic acid molecule of the invention, but which does not substantially recognize and bind other molecules in a sample, for example, a biological sample, which naturally includes a protein of the invention.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “vector” is meant a nucleic acid molecule, for example, a plasmid, cosmid, or bacteriophage, that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

### BRIEF DESCRIPTION OF THE DRAWINGS

FIGS. 1A-1D are Northern blots showing the identification of Kras regulated miRNAs and expression of miR-143/145 in Pancreatic ductal adenocarcinoma (PDAC). FIG. 1A provides a series of five Northern blots demonstrating miRNA expression in HPNE and HPNE-Kras^{G12D}. FIG. 1B provides a series of six Northern blots showing expression of miR-143 and miR-145 in HPNE and 15 PDAC cell lines. FIG. 1C provides a series of three Northern blots showing expression of miR-143 and miR-145 in tissues isolated from normal pancreas and low passage xenografts. FIG. 1D provides a series of three Northern blots showing expression of miR-143 and miR-145 in the mouse fibroblast derived-NIH3T3 and NIH3T3-K-Molv cell lines. U6 served as a loading control.

FIGS. 2A and 2B show the structure and sequence of the miR-143/145 pri-miRNA transcript. FIG. 2A is a schematic diagram. Shown is the structure of the miR-143/145 pri-miRNA transcript as defined by 5′ and 3′ RACE. The plot shown below the transcript represents evolutionary conservation taken from the UCSC Genome Browser (human genome May 2004 assembly). Shown is the location of a RREB-1 binding site within this genomic region. FIG. 2B provides the DNA sequence of the spliced miR-143/145 pri-miRNA transcript.

FIGS. 3A-3C show that RREB-1 negatively regulates expression of miR-143/145. FIG. 3A is a graph showing the phylogenetic conservation of the miR-143/145 promoter region. Vista was used to generate pairwise alignments between the genomic sequence from human (May 2004 assembly) and the indicated species. The graph is a plot of nucleotide identity for a 100-bp sliding window centered at a given position. Arrow indicates the location of a RREB-1 binding site which has been annotated within the first exon of the miR-143/145 pri-miRNA transcript (top). The RREB-1 binding site sequence at this region is contrasted to the RREB-1 consensus sequence (bottom). FIG. 3B is a graph showing the results of a quantitative RT-PCR analysis of RREB-1 transcript expression and miR-143/145 pri-miRNA transcript expression in HPNE, HPNE-Kras^{G12D} or HPNE-Kras^{G12D} cells infected with RREB-1-shRNA lentivirus or with a control-shRNA lentivirus. Expression of RNA transcripts was normalized to β-actin. FIG. 3C is a graph showing the results of a quantitative RT-PCR analysis of miR-143 and miR-145 expression in the cell lines described above. miRNA expression was normalized to 18S RNA. Error bars represent standard deviations.

FIGS. 4A-4D shows that miR-143/145 expression is restituted by inhibition of the RAF/MEK/MAPK pathway. FIG. 4A provides a series of Northern blots demonstrating miRNA expression in HPNE-Kras^{G12D} treated with the P13K inhibitor-LY294002 or the MAPK inhibitor-U0126. U6 served as a loading control. FIG. 4B is a graph showing the results of a quantitative RT-PCR analysis of miR-143 and miR-145 expression in HPNE, and HPNE-Kras^{G12D} treated with LY294002 or U0126. miRNA expression was normalized to 18S RNA. FIG. 4C is a graph showing the results of a quantitative RT-PCR analysis of miR-143/145 pri-mRNA transcript expression in HPNE-Kras^{G12D} treated with LY294002 or U0126. pri-mRNA transcript expression was normalized to β-actin. Error bars represent standard deviations. FIG. 4D is a graph showing the results of a quantitative RT-PCR analysis of RREB-1 expression in HPNE-Kras^{G12D} treated with U0126.

FIGS. 5A-5F show that miR-143/145 influences the transformed phenotype of pancreatic cancer cells. FIG. 5A provides a Northern blot analysis demonstrating stable expression of miR-143 and miR-145 in HPNE-Kras^{G12D}, MiaPaCa-2 and Panc1. U6 served as a loading control. FIG. 5B provides three graphs showing the results of a colorimetric cell growth assay (CKK-8, Dojindo) that was used to monitor growth rates of cells infected with retroviruses. FIG. 5C shows the anchorage-independent growth of retrovirally-infected HPNE-Kras^{G12D}, MiaPaCa-2, and Panc1 cells was assayed by growth in soft agar. FIG. 5D provides photographs of nude mouse injected with MiaPaCa-2 cells infected with the miR-143/145 retrovirus (left) or with an empty retrovirus (right). Small panels reveal the dissected injection sites. The animal shown is representative of five mice all of which showed identical results. The graph summarizes the results of similar experiments performed with the Panc1 cell line. FIG. 5E shows a Northern blot analysis demonstrating stable expression of either miR-143 or miR-145 in MiaPaCa-2. U6 served as a loading control. FIG. 5F provides two graphs showing the results of a colorimetric cell growth assay (CKK-8, Dojindo) that was used to monitor growth rates of MiaPaCa-2 cells infected with retroviruses. FIG. 5G provides three micrographs showing anchorage-independent growth of retrovirally-infected MiaPaCa-2 cells expressing either miR-143 or miR-145 assayed by growth in soft agar. FIG. 5H provides photographs of a nude mice injected with MiaPaCa-2 cells infected with the miR-143 retrovirus (left) or with miR-145 retrovirus (right). A nude mouse injected with MiaPaCa-2 cells infected an empty retrovirus was used as a control. The animals shown are representative of five mice all of which showed identical results. The graph shows the change in average tumor volume measured over 30 day interval.

FIG. 6 shows sequences encoding miR-143, miR-145 or both together (miR-143/145) were cloned into the XhoI restriction site within the multiple cloning site of the commercially-available vector pMSCV-Neo from Clontech.

### DETAILED DESCRIPTION OF THE INVENTION

The invention features compositions and methods that are useful for the diagnosis, treatment or prevention of a neoplasia.

The invention is based, at least in part, on the discoveries that Kras upregulated miR-34a, miR-199b and miR-31 and downregulated miR-27b and the miR-143/145 cluster in a cell line with enforced expression of activated Kras. Reduced levels of miR-143 and miR-145 were also observed in pancreatic ductal adenocarcinoma cell lines and in neoplastic cells derived from patients with pancreatic cancer. Surprisingly, expression of miR-143/145 dramatically inhibited anchorage-independent growth of neoplastic cells and infection by a miR-143/145 virus completely abrogated tumor formation when these cells were injected into nude mice. Accordingly, the invention provides compositions and methods for altering the expression of a microRNA of the invention thereby treating a neoplasia. Desirably, the invention provides agents that downregulate miR-34a, miR-199b and miR-31 and that upregulate miR-27b and the miR-143/145 cluster. For diagnostic purposes, a decrease in MAPK signaling indicates that the subject will benefit from a therapy described herein. This is in view of the discovery reported below that increased Kras signaling leads to an increase in MAPK signaling. Thus, where a neoplasia is characterized by increased Kras or MAPK signaling, agents that increase levels of miR-27b and the miR-143/145 are useful for the treatment of the neoplasia. Such agents are generally useful for the treatment of neoplasia, particularly of pancreatic cancers, including pancreatic ductal adenocarcinoma.

### Inhibitory Nucleic Acid Molecules

Given that Kras upregulation of miR-34a, miR-199b and miR-31 is associated with cancer, the invention provides compositions that inhibit the expression of these microRNAs as well as methods of using such compositions for the treatment of cancer. In one embodiment, the invention provides inhibitory nucleic acid molecules, such as antisense, siRNA, or shRNA nucleic acid molecules, that decrease the expression of miR-34a, miR-199b or miR-31. Inhibitory nucleic acid molecules are essentially nucleobase oligomers that may be employed to decrease the expression of a target nucleic acid sequence, such as a nucleic acid sequence that encodes a of miR-34a, miR-199b and miR-31 microRNA. The inhibitory nucleic acid molecules provided by the invention include any nucleic acid molecule sufficient to decrease the expression of a miR-34a, miR-199b and miR-31 by at least 5-10%, desirably by at least 25%-50%, or even by as much as 75%-100%. Each of the nucleic acid sequences provided herein may be used, for example, in the discovery and development of therapeutic inhibitory nucleic acid molecules to decrease the expression of miR-34a, miR-199b or miR-31. If desired, inhibitory nucleic acid molecules that target miR-34a, miR-199b or miR-31 are administered in combination, such that the coordinated reduction in the expression of two or more microRNAs is achieved.

The invention encompasses virtually any single-stranded or double-stranded nucleic acid molecule that decreases expression of a miR-34a, miR-199b or miR-31 microRNA. The invention further provides catalytic RNA molecules or ribozymes. Such catalytic RNA molecules can be used to inhibit expression of a microRNA nucleic acid molecule in vivo.

The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif Examples of such hammerhead motifs are described by Rossi et al., Nucleic Acids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In another approach, the inhibitory nucleic acid molecule is a double-stranded nucleic acid molecule used for RNA interference (RNAi)-mediated knock-down of the expression of a microRNA. siRNAs are also useful for the inhibition of microRNAs. See, for example, Nakamoto et al., Hum Mol Genet, 2005. Desirably, the siRNA is designed such that it provides for the cleavage of a target microRNA of the invention. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to a miR-34a, miR-199b or miR-31 comprises at least a fragment of the double-stranded gene, such that each strand of the double-stranded inhibitory nucleic acid molecule is capable of binding to the complementary strand of the target gene. The inhibitory nucleic acid molecule need not have perfect correspondence or need not be perfectly complementary to the reference sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

Inhibitory nucleic acid molecules of the invention also include double stranded nucleic acid “decoys.” Decoy molecules contain a binding site for a transcription factor that is responsible for the deregulated transcription of a gene of interest. The present invention provides decoys that competitively block binding to a regulatory element in a target gene (e.g., miR-34a, miR-199b or miR-31). The competitive inhibition of binding by the decoy results in the indirect inhibition of transcription of a target microRNA. An overview of decoy technology is provided by Suda et al., Endocr. Rev., 1999, 20, 345-357; S. Yla-Hertttuala and J. F. Martin, The Lancet 355, 213-222, 2000). In one therapeutic method, short double-stranded DNA decoy molecules are introduced into cells (e.g., neoplastic cells) of a subject. The decoys are provided in a form that facilitates their entry into target cells of the subject. Having entered a cell, the decoy specifically binds an endogenous transcription factor, thereby competitively inhibiting the transcription factor from binding to an endogenous gene. The decoys are administered in amounts and under conditions whereby binding of the endogenous transcription factor to the endogenous gene is effectively competitively inhibited without significant host toxicity. Depending on the transcription factor, the methods can effect up- or down-regulation of gene expression. The subject compositions comprise the decoy molecules in a context that provides for pharmacokinetics sufficient for effective therapeutic use.

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient having a neoplasia receives a dosage between about 50 and 300 mg/m²/day (e.g., 50, 75, 100, 125, 150, 175, 200, 250, 275, and 300).

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with a nucleic acid molecule of a microRNA described herein. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In other embodiments, a single stranded modified nucleic acid molecule (e.g., a nucleic acid molecule comprising a phosphorothioate backbone and 2′-O-Me sugar modifications is conjugated to cholesterol. Such conjugated oligomers are known as “antagomirs.” Methods for silencing microRNAs in vivo with antagomirs are described, for example, in Krutzfeldt et al., Nature 438: 685-689.

### Polynucleotides of the Invention

In general, the invention includes any nucleic acid sequence encoding a microRNA described herein (e.g., miR-143, miR-145, miR-27b, miR-31, miR-34a and/or miR-199b) as well as nucleic acid molecules containing at least one strand that hybridizes with a nucleic acid sequence of miR-143, miR-145, miR-27b, miR-31, miR-34a and/or miR-199b (e.g., an inhibitory nucleic acid molecule, such as an antisense molecule, a dsRNA, siRNA, or shRNA). The inhibitory nucleic acid molecules of the invention can be between 8 and 45 nucleotides in length. In some embodiments, the inhibitory nucleic acid molecules of the invention comprises 8, 10, 15, 16, 17, 18, 19, 20, 21, 22, 23, 24, 25, 26, 27, 28, 29, 30, 31, 32, 33, 34, 35, 45, or complementary nucleotide residues. In yet other embodiments, the antisense molecules are 60%, 65%, 70%, 75%, 80%, 85%, 90%, 95%, 96%, 97%, 98%, 99% complementary to the target sequence. An isolated nucleic acid molecule can be manipulated using recombinant DNA techniques well known in the art. Thus, a nucleotide sequence contained in a vector in which 5′ and 3′restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### Delivery of Nucleobase Oligomers

Naked oligonucleotides are capable of entering tumor cells and inhibiting the expression of a target microRNA (e.g., miR-34a, miR-199b or miR-31). Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Polynucleotide Therapy

Given that Kras downregulation of miR-27b and the miR-143/145 cluster is associated with neoplasia, the invention provides polynucleotide therapy useful for increasing the expression of these microRNAs. The invention also provides polynucleotide therapy featuring vectors encoding inhibitory nucleic acid molecules or analogs thereof that target a microRNA delineated herein (i.e., a Kras upregulated microRNA, such as miR-34a, miR-199b and miR-31). Expression vectors encoding a desired sequence (e.g. encoding an inhibitory nucleic acid molecule or a microRNA) can be delivered to cells of a subject having a neoplasia. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an inhibitory nucleic acid molecule can be cloned into a retroviral vector and expression can be driven from its endogenous promoter, from the retroviral long terminal repeat, or from a promoter specific for a target cell type of interest. A similar strategy can be used to express a microRNA to increase its expression. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of a therapeutic nucleic acid molecule to a cell of a patient diagnosed as having a neoplasia. For example, an inhibitory nucleic acid molecule that targets a miR-34a, miR-199b or miR-31 or an expression vector that encodes a miR-143, miR-145, miR-27b microRNA can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Nucleic acid molecule expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. Pancreatic cancer may be treated, for example, by expressing miR-27b and the miR-143/145 cluster using a tissue-specific or ubiquitously expressed promoter. Tissue specific promoters useful for the treatment of pancreatic cancer include, but are not limited to, the mesothelin promoter (Showalter et al., Cancer Biol Ther. 2008 October; 7(10):1584-90), and other promoters expressed in pancreatic cancer cells. Cancer-specific promoters are described, for example, in U.S. Patent Publication No. 20080286860, which is incorporated herein by reference in its entirety. For example, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, agents that downregulate miR-34a, miR-199b and miR-31 and that upregulate miR-27b and the miR-143/145 cluster are useful alone, or in any combination, for the treatment of cancer. Furthermore, the invention provides agents that reduce Kras or Map Kinase pathway activity. In particular, the invention provides therapeutic compositions that alter the expression of microRNAs whose levels are perturbed by Kras activation. In one embodiment, the present invention provides a pharmaceutical composition for the treatment of neoplasia (e.g., pancreatic cancer) comprising an effective amount of U0126. The structure of U0126 is provided below.

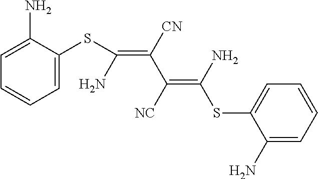

MAPK inhibitors useful in the methods of the invention include but are not limited to PD98059 [2′-amino-3′-methoxyflavone](Calbiochem Corp., La Jolla, Calif.) and PD184352 (CI-1040) [2-(2-chloro-4-iodo-phenylamino)-Ncyclopropylmethoxy-3,4-difluoro-benzamide] and U0126 [1,4-diamino-2,3-dicyano-1,4-bis(2-aminophenylthio)butadiene].

In other embodiments, the invention provides pharmaceutical compositions comprising an inhibitory nucleic acid molecule (e.g., an antisense, siRNA, or shRNA polynucleotide) that decreases the expression of one or more of miR-34a, miR-199b or miR-31. In another embodiment, the invention provides a pharmaceutical composition comprising an expression vector encoding miR-27b, miR-143 or miR145 microRNA or comprising miR-27b, miR-143 or miR145 microRNAs to treat neoplasia. If desired, such nucleic acid molecules are administered in combination with a chemotherapeutic agent. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

Polynucleotides of the invention may be administered within a pharmaceutically-acceptable diluent, carrier, or excipient, in unit dosage form. Conventional pharmaceutical practice may be employed to provide suitable formulations or compositions to administer the compounds to patients suffering from a neoplasia (e.g., cancer). Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase oligomer of the invention is likely to depend on such variables as the type and extent of the disorder, the overall health status of the particular patient, the formulation of the compound excipients, and its route of administration.

With respect to a subject having a neoplastic disease or disorder, an effective amount is sufficient to stabilize, slow, or reduce the proliferation or metastasis of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a therapeutic polynucleotide.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. As described above, if desired, treatment with a therapeutic polynucleotide or an inhibitory nucleic acid molecule of the invention may be combined with therapies for the treatment of proliferative disease (e.g., radiotherapy, surgery, or chemotherapy). For any of the methods of application described above, an inhibitory nucleic acid molecule of the invention is desirably administered intravenously or is applied to the site of neoplasia (e.g., by injection).

### Diagnostics

As described in more detail below, the present invention has identified alterations in expression levels of miR-34a, miR-199b, miR-31, miR-27b and the miR-143/145 cluster that are associated with cancer. Such alterations in microRNA expression have been observed in pancreatic cancer cells, as well as other cells where Kras is activated. Thus, alterations in the expression level of one or more (e.g., 1, 2, 3, 4, 5, 6, 7, 8, or 9) of the following markers is used to diagnose a neoplasia: activated Kras, increased levels or biological activity of a MAPK signaling pathway component, upregulated miR-34a, miR-199b and miR-31 and down-regulated miR-27b and the miR-143/145 cluster. If desired, alterations in the expression of all of these markers is used to diagnose or characterize a neoplasia.

In one embodiment, a subject is diagnosed as having or having a propensity to develop a neoplasia, the method comprising measuring markers in a biological sample from a patient, and detecting an alteration in the expression of test marker molecules relative to the sequence or expression of a reference molecule. The markers typically include miR-143, miR-145, miR-27b, miR-31, miR-34a and/or miR-199b. While the following approaches describe diagnostic methods featuring a miR-143, miR-145, miR-27b, miR-31, miR-34a and/or miR-199b microRNA, the skilled artisan will appreciate that any one or more of the markers delineated herein is useful in such diagnostic methods. Alterations in microRNA expression are detected using methods known to the skilled artisan and described herein. Such information can be used to diagnose a neoplasia.

In one approach, diagnostic methods of the invention are used to assay the expression of a miR-143, miR-145, miR-27b, miR-31, miR-34a and/or miR-199b in a biological sample relative to a reference (e.g., the level of microRNA present in a corresponding control tissue). In one embodiment, the level of a miR-143, miR-145, miR-27b, miR-31, miR-34a and/or miR-199b is detected using a nucleic acid probe that specifically binds the microRNA. By “nucleic acid probe” is meant any nucleic acid molecule, or fragment thereof, that binds a microRNA. Such nucleic acid probes are useful for the diagnosis of a neoplasia. The specificity of the probe determines whether the probe hybridizes to a naturally occurring sequence, allelic variants, or other related sequences. Hybridization techniques may be used to identify mutations indicative of a neoplasia or may be used to monitor expression levels of these genes (for example, by Northern analysis (Ausubel et al., supra).

In another approach, quantitative PCR methods are used to identify an alteration in the expression of a miR-143, miR-145, miR-27b, miR-31, miR-34a and/or miR-199b. In another approach, PCR methods are used to identify an alteration in the sequence of a microRNA.

In general, the measurement of a nucleic acid molecule in a subject sample is compared with a diagnostic amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant increase or decrease (e.g., at least about 5%, 10%, 15%, 30%, 50%, 60%, 75%, 80%, or 90%) in the level of test nucleic acid molecule or polypeptide in the subject sample relative to a reference may be used to diagnose a neoplasia. Test molecules include any one or more of miR-143, miR-145, miR-27b, miR-31, miR-34a and/or miR-199b. In one embodiment, the reference is the level of test polypeptide or nucleic acid molecule present in a control sample obtained from a patient that does not have a neoplasia. In another embodiment, the reference is a baseline level of test molecule present in a biologic sample derived from a patient prior to, during, or after treatment for a neoplasia. In yet another embodiment, the reference can be a standardized curve.

### Microarrays

The nucleic acid molecules of the invention (e.g., miR-34a, miR-199b, miR-31, miR-27b, miR-143, miR-145, and polynucleotides encoding such microRNAs) are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or microRNAs. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

To produce a nucleic acid microarray, oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. microRNA, RNA or DNA) derived from a biological sample may be used to produce a hybridization probe as described herein. The biological samples are generally derived from a patient, preferably as a bodily fluid (such as blood, cerebrospinal fluid, phlegm, saliva, or urine) or tissue sample (e.g. a tissue sample obtained by biopsy). For some applications, cultured cells (e.g., neoplastic cells) or other tissue preparations may be used. Alterations in the levels of the microRNA (e.g., mature microRNA, microRNA precursory, or primary transcript) are then analysed. Patient samples having increases in the level of miR-34a, miR-199b and miR-31 or reductions in the level of miR-27b and the miR-143/145 are identified according to the methods described herein.

The mRNA encoding a microRNA is isolated according to standard methods, and cDNA is produced and used as a template to make complementary RNA suitable for hybridization. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. Useful variations of hybridization conditions will be readily apparent to those skilled in the art. A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Microarrays comprising a miR-34a, miR-199b, miR-31, miR-27b, miR-143, miR-145 polynucleotide or a polynucleotide that specifically binds to such a microRNA are particularly useful in the diagnostic methods of the invention.

### Types of Biological Samples

The level of markers in a biological sample from a patient having or at risk for developing a neoplasia can be measured, and an alteration in the expression of test marker molecule relative to the sequence or expression of a reference molecule, can be determined in different types of biologic samples. Test markers include any one or all of the following: mir-miR-143, miR-145, miR-27b, miR-31, miR-34a, miR-199b, Kras, component of a MAP kinase pathway. The biological samples are generally derived from a patient, preferably as a bodily fluid (such as blood, cerebrospinal fluid, phlegm, saliva, or urine) or tissue sample (e.g. a tissue sample obtained by biopsy).

### Therapy Selection

After a subject is diagnosed as having a neoplasia (e.g., pancreatic cancer) a method of treatment is selected. In pancreatic cancer, for example, a number of standard treatment regimens are available. As described herein, identification of alterations in the expression of particular microRNAs or Kras activation is used in selecting a treatment method. MicroRNA expression profiles that correlate with poor clinical outcomes (e.g., upregulation of miR-34a, miR-199b and miR-31 and downregulation of miR-27b and the miR-143/145) are identified as aggressive neoplasias. An aggressive neoplasia is typically associated with a poorer prognosis (i.e., a higher probability of metastasis and/or death). MicroRNA expression profiles that correlate with good clinical outcomes are identified as less aggressive neoplasias.

Less aggressive neoplasias are likely to be susceptible to conservative treatment methods. More aggressive neoplasias are less susceptible to conservative treatment methods. Conservative treatment methods include, for example, chemotherapy using agents at dosages that are unlikely to cause adverse side effects, surgery that minimizes damage to tissues adjoining a tumor, or radiotherapy at a dosage that is likely to slow tumor growth without causing adverse side effects. Alternatively, a conservative treatment method might include cancer surveillance, which involves periodic patient monitoring using diagnostic assays. More aggressive neoplasias are treated with higher doses of chemotherapeutic or radiotherapeutic agents, with chemotherapeutic agents having increased toxicity, or with more radical surgery.

### Kits

The invention provides kits for the diagnosis or monitoring of a neoplasia, such as pancreatic ductal adenocarcinoma. In one embodiment, the kit detects an alteration in the expression of a Marker (e.g., miR-143, miR-145, miR-27b, miR-31, miR-34a and/or miR-199b) relative to a reference level of expression. In another embodiment, the kit detects an alteration in the sequence of a miR-143, miR-145, miR-27b, miR-31, miR-34a and/or miR-199b derived from a subject relative to a reference sequence. In related embodiments, the kit includes reagents for monitoring the expression of a miR-143, miR-145, miR-27b, miR-31, miR-34a and/or miR-199b nucleic acid molecule, such as primers or probes that hybridize to a miR-143, miR-145, miR-27b, miR-31, miR-34a and/or miR-199b nucleic acid molecule.

Optionally, the kit includes directions for monitoring the nucleic acid molecule levels of a Marker in a biological sample derived from a subject. In other embodiments, the kit comprises a sterile container which contains the primer, probe, antibody, or other detection regents; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a neoplasia can be monitored using the methods and compositions of the invention. In one embodiment, the disease state of a patient can be monitored using the methods and compositions of the invention. Such monitoring may be useful, for example, in assessing the efficacy of a particular drug in a patient. Therapeutics that alter the expression of any one or more of the Markers of the invention (e.g., Kras activity, Map Kinase pathway signalling, miR-143, miR-145, miR-27b, miR-31, miR-34a and/or miR-199b) are taken as particularly useful in the invention.

### Screening Assays

One embodiment of the invention encompasses a method of identifying an agent that inhibits or increases the expression or activity of a miR-143, miR-145, miR-27b, miR-31, miR-34a and/or miR-199b microRNA. Accordingly, compounds that modulate the expression or activity of a miR-143, miR-145, miR-27b, miR-31, miR-34a and/or miR-199b nucleic acid molecule, variant, or portion thereof are useful in the methods of the invention for the treatment or prevention of a neoplasm (e.g., prostate cancer). The method of the invention may measure a decrease in transcription of one or more microRNAs of the invention. Any number of methods are available for carrying out screening assays to identify such compounds. In one approach, the method comprises contacting a cell that expresses a microRNA with an agent and comparing the level of microRNA expression in the cell contacted by the agent with the level of expression in a control cell, wherein an agent that reduces the expression of a miR-34a, miR-199b and miR-31 microRNA or increases the expression of a miR-27b, miR-143, or miR-145 cluster, thereby inhibits a neoplasia. In another approach, candidate compounds are identified that specifically bind to and alter the activity of a microRNA of the invention. Methods of assaying such biological activities are known in the art and are described herein. The efficacy of such a candidate compound is dependent upon its ability to interact with a miR-143, miR-145, miR-27b, miR-31, miR-34a and/or miR-199b microRNA. Such an interaction can be readily assayed using any number of standard binding techniques and functional assays (e.g., those described in Ausubel et al., supra).

Potential agonists of a miR-27b miR-143, or miR145 microRNA include any agent that enhances the expression or biological activity of the microRNA. Potential antagonists of a miR-31, miR-34a and/or miR-199b microRNA include agents that bind to a nucleic acid sequence of the invention and thereby reduce or extinguish its activity. Such agents include organic molecules, peptides, peptide mimetics, polypeptides, nucleic acid molecules, such as double-stranded RNAs, siRNAs, antisense polynucleotides, and antibodies. Also included are small molecules that bind to the microRNA thereby altering binding to cellular molecules with which the microRNA normally interacts, such that the normal biological activity of the microRNA is altered (i.e., increased or decreased). Small molecules of the invention preferably have a molecular weight below 2,000 daltons, more preferably between 300 and 1,000 daltons, and still more preferably between 400 and 700 daltons. It is preferred that these small molecules are organic molecules.

Compounds that are identified as binding to a miR-143, miR-145, miR-27b, miR-31, miR-34a and/or miR-199b microRNA of the invention with an affinity constant less than or equal to 10 mM are considered particularly useful in the invention. Alternatively, any in vivo protein interaction detection system, for example, any two-hybrid assay may be utilized to identify compounds that interact with such microRNAs. Interacting compounds isolated by this method (or any other appropriate method) may, if desired, be further purified (e.g., by high performance liquid chromatography). Compounds isolated by any approach described herein may be used as therapeutics to treat a neoplasia in a human patient.

In addition, compounds that normalize the expression of miR-143, miR-145, miR-27b, miR-31, miR-34a and/or miR-199b, whose expression is altered in a subject having a neoplasia are also useful in the methods of the invention. Any number of methods are available for carrying out screening assays to identify new candidate compounds that increase or decrease the expression of a miR-143, miR-145, miR-27b, miR-31, miR-34a and/or miR-199b microRNA.

The invention also includes novel compounds identified by the above-described screening assays. Optionally, such compounds are characterized in one or more appropriate animal models to determine the efficacy of the compound for the treatment of a neoplasia. Desirably, characterization in an animal model can also be used to determine the toxicity, side effects, or mechanism of action of treatment with such a compound. Furthermore, novel compounds identified in any of the above-described screening assays may be used for the treatment of a neoplasia in a subject. Such compounds are useful alone or in combination with other conventional therapies known in the art.

### Test Compounds and Extracts

In general, compounds capable of inhibiting the growth or proliferation of a neoplasia by altering the expression or biological activity of a miR-143, miR-145, miR-27b, miR-31, miR-34a and/or miR-199b are identified from large libraries of either natural product or synthetic (or semi-synthetic) extracts or chemical libraries according to methods known in the art. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptide libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

In an embodiment of the invention, a high thoroughput approach can be used to screen different chemicals for their potency to affect the activity of a miR-143, miR-145, miR-27b, miR-31, miR-34a and/or miR-199b microRNA. For example, a cell based sensor approach can be used to identify agents that inhibit expression of miR-143, miR-145, miR-27b, miR-31, miR-34a and/or miR-199b. In one embodiment, the invention provides a method for identifying an agent that inhibits a neoplasia, the method comprising contacting a cell containing a sensor construct with an agent (polynucleotide, polypeptide, or small molecule), where the sensor construct contains a reporter gene linked to a site complementary to a microRNA of the invention; and measuring an alteration in the expression of the reporter gene relative to the expression of the reporter gene present in a control vector (e.g., a control vector not having a site complementary to the microRNA), wherein an alteration in the level of reporter expression identifies the agent as treating a neoplasia.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of a microRNA (e.g., miR-143, miR-145, miR-27b, miR-31, miR-34a and/or miR-199b) variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, compounds shown to be useful agents for the treatment of a neoplasm are chemically modified according to methods known in the art.

Accordingly, the present invention provides methods of treating disease and/or disorders or symptoms thereof which comprise administering a therapeutically effective amount of a pharmaceutical composition comprising an agent of the formulae herein to a subject (e.g., a mammal such as a human). Thus, one embodiment is a method of treating a subject suffering from or susceptible to a neoplastic disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which Kras activity activation or activation of the Map Kinase (MAPK) cascade (also known as the RAF/MEK/MAPK pathway) may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasia, particularly neoplasias characterized as having increased Kras activity, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Combination Therapies

Optionally, any therapeutic delineated herein may be administered in combination with a standard anti-neoplasia therapy; such methods are known to the skilled artisan and described in Remington's Pharmaceutical Sciences by E. W. Martin. Exemplary anti-neoplastic therapies include, for example, chemotherapy, cryotherapy, hormone therapy, radiotherapy, and surgery. Combinations of the invention provide for the administration of a therapeutic polynucleotide or inhibitory nucleic acid described herein (e.g., an inhibitory nucleic acid molecule or other agent that inhibits the expression of miR-34a, miR-199b and miR-31 microRNA or a nucleotide encoding miR-27b and the miR-143/145 cluster) alone or in any combination. Specifically, any method known in the art that increases the expression of mir-27b and/or mir-143/145 is expected to be therapeutic. Similarly, any method that lowers the expression of mir-34a, mir-199b, and mir-31 is expected to be therapeutic. If desired the combination includes one, two, three, four, five or even six therapeutics that reduce or increase the levels of microRNAs defined herein or that modulates Kras or MapK signalling.

A therapeutic regimen of the invention may, if desired, include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Kras Upregulated miR-34a, miR-199b and miR-31 and Downregulated miR-27b and the miR-143/145 Cluster

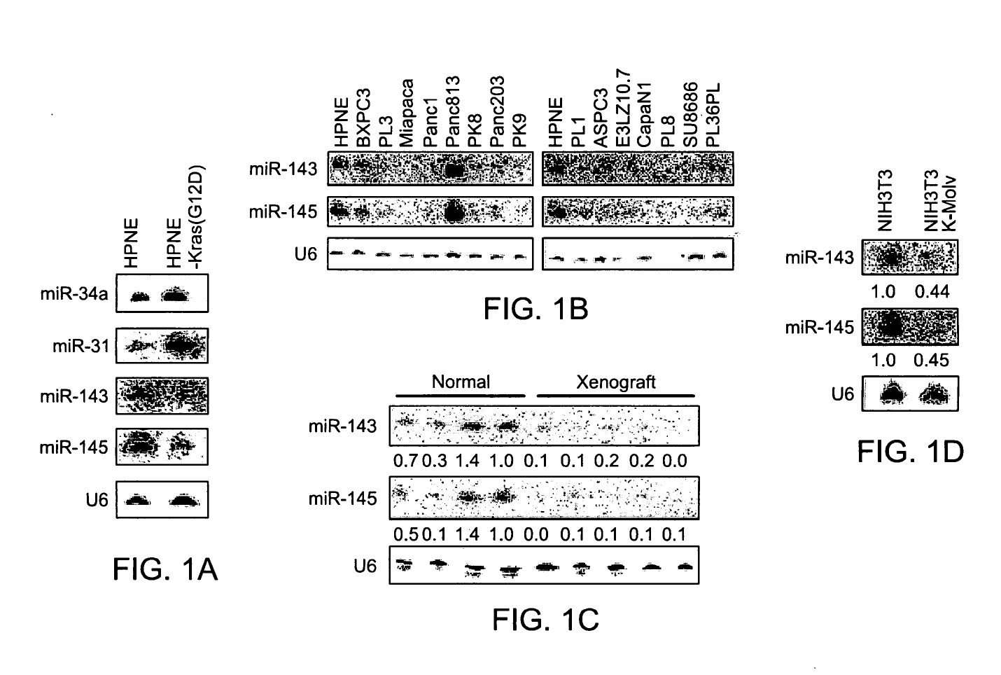

Pancreatic ductal adenocarcinoma (PDAC) is one of the most lethal human malignancies. Mutational activation of the KRAS2 oncogene occurs in over 90% of PDAC cases. In the vast majority of cases, codon 12 is the target of these mutations. In order to determine whether constitutively active Kras signaling influences miRNA expression, a custom microarray was used to examine global miRNA expression profiles in the non-transformed pancreatic ductal epithelial cell line HPNE and a paired cell line with enforced expression of mutant Kras) (Kras^{G12D}. Kras signaling in HPNE led to upregulation of 3 miRNAs (miR-34a, miR-199b and miR-31) and downregulation of miR-27b and the miR-143/145 cluster (Table 1 and FIG. 1a).

TABLE 1
miRNAs identified as up or downregulated in
HPNE-Kras^{(G12D)} versus HPNE.

| | | | HPNE- |
|---|---|---|---|
| miRNA: | HPNE | HPNE-Kras^{(G12D)} | Kras^{(G12D)}/HPNE |
| miR-34a | 285 | 981 | 3.4 |
| miR-199b | 52 | 161 | 3.1 |
| miR-31 | 343 | 983 | 2.9 |
| miR-27b | 181 | 87 | 0.48 |
| miR-145 | 1513 | 352 | 0.23 |
| miR-143 | 443 | 60 | 0.14 |

Northern blotting was used to validate a subset of these miRNA expression changes in HPNE and HPNE-Kras^{G12D} cell lines (FIG. 1A). Regulation of these miRNAs by Kras is likely to have important consequences for neoplastic transformation given that miR-34a is a critical downstream component of p53 with potent anti-proliferative and pro-apoptotic activity (Chang et al., 2007; He et al., 2007). Furthermore, the Myc oncogene directly down-regulates miR-34a (Chang et al., 2008). It is likely that upregulation of miR-34a in the setting of activated Kras is a secondary consequence of oncogene-induced senescence, a p53-dependent response to Kras activation that often occurs in nontransformed cells (Sherr and Weber, 2000). Significantly less is known about miR-31, although this miRNA has been observed to be overexpressed in colorectal carcinoma cell lines which frequently harbor KRAS mutations (Bandres, et al., 2006).

### Example 2

### miR-143 and miR-145 Levels were Reduced in PDAC Cell Lines and Pancreatic Cancers

Of particular interest is the miR-143/145 cluster. Decreased expression of miR-143 and miR-145 is a frequent feature of colorectal and breast tumors (Iorio, M. V., et al. 2005, Michael et al., 2003). Moreover, these miRNAs exhibit decreased expression in a variety of cancer cell lines including those derived from breast, lung, prostate, ovarian, and lymphoid cancers. Using northern blotting, miR-143 and miR-145 were found to be frequently expressed at low levels in PDAC cell lines as compared to HPNE cells (FIG. 1B). In addition, northern blotting demonstrated decreased expression of miR-143/145 in low-passage xenografts established directly from patients with pancreatic cancer (FIG. 1C). This regulation was not limited to PDAC since similar regulation was observed in a mouse fibroblast cell line with enforced oncogenic Kras expression (FIG. 1D). These findings suggest a general mechanism whereby Kras signaling leads to altered expression of these miRNAs in broad range of cell types and tumor types.

### Example 3

### A Kras-RREB-1 Signaling Pathway Represses miR-143/145 Expression

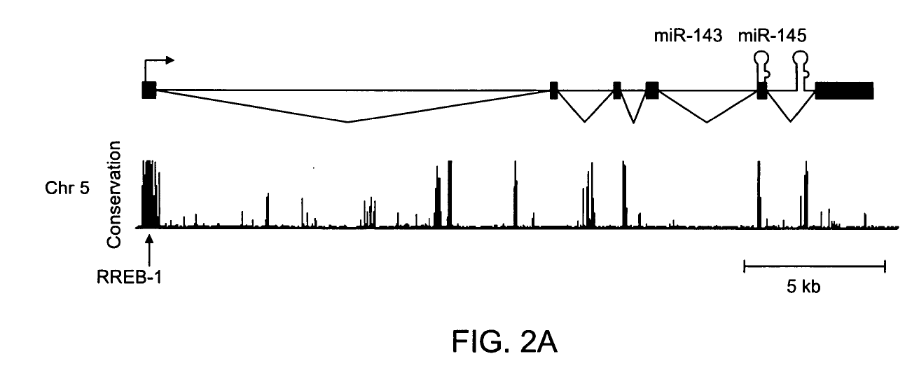

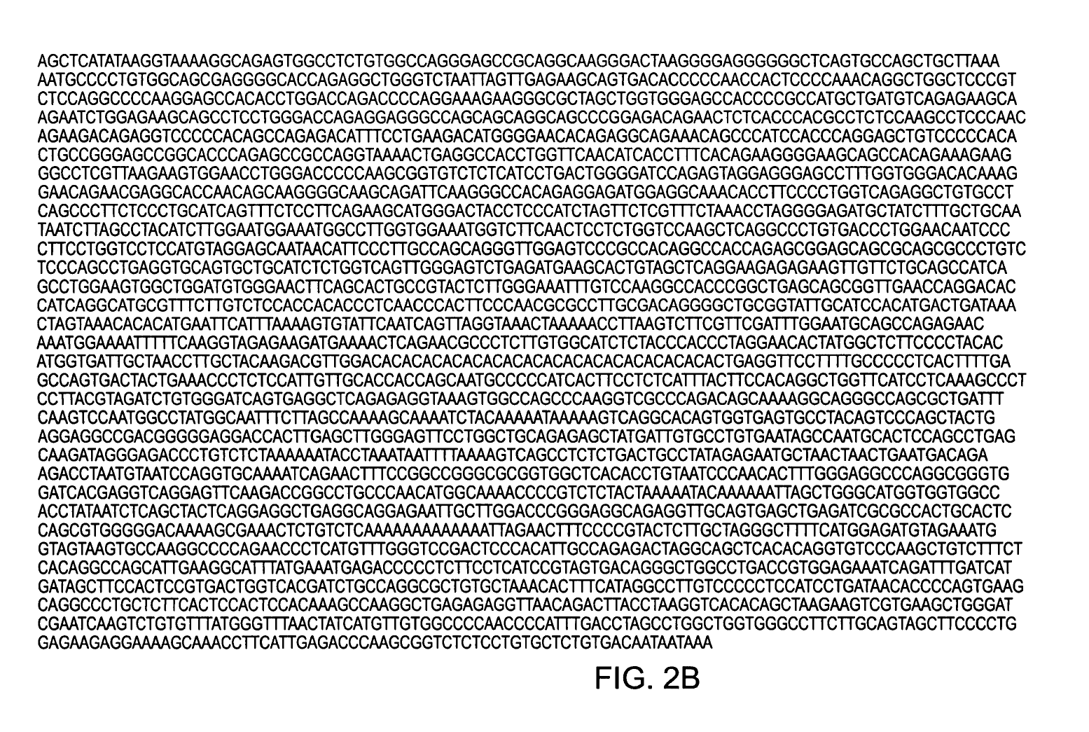

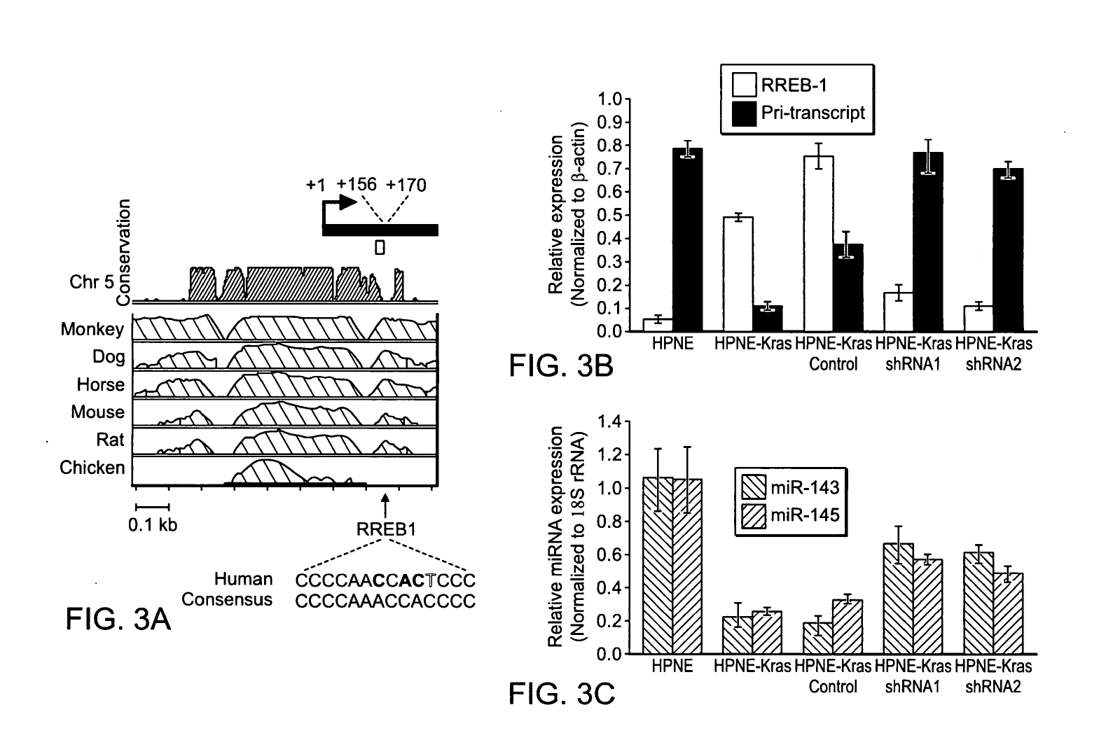

In order to investigate how Kras signaling downregulates miR-143/145, the structure of the single primary transcript (pri-miRNA) that encodes both of these miRNAs was characterized. Using a combination of 5′ and 3′ rapid amplification of cDNA ends (RACE), an approximately 26 kb primary transcript was mapped that is spliced to a 3 kb transcript that encodes miR-143 and miR-145 (FIGS. 2A and 2B). Interestingly, miR-143 is located in an exon consisting almost exclusively of the pre-miRNA sequence and miR-145 is located in the adjacent intron (FIG. 2a). Consistent with transcriptional repression of the miR-143/145 cluster, this primary transcript exhibits reduced expression in HPNE-Kras^{G12D} cells compared to HPNE cells (FIG. 3b).

The miR-143/145 pri-miRNA transcript has a highly conserved transcription start site containing a Ras responsive element (RRE) in the first exon (FIG. 3A). RREs have previously been demonstrated to be a binding site for the Ras responsive element binding protein-1 (RREB-1), a transcription factor which is known to act as both an activator and repressor of gene expression in response to Ras pathway activity and has been implicated as a potential human oncogene (Date et al., 2004; Mukhipadhyay et al., 2007; Oxford et al., 2007; Thiagalingam et al., 1996; Thiagalingam et al., 1997; Uren et al., 2008; Zhang et al., 2003). The RREB-1 transcription factor has been shown to augment the Ras transcriptional activation of the calcitonin (CT) gene and negatively regulate a specific p16 promoter containing a point mutation in BALB/c mice (Thiagalingam et al., 1996; Zhang et al., 2003).

To test the hypothesis that the RRE in the miR-143/145 promoter would bind RREB-1, leading to repression of these miRNAs, miR-143/145 expression in HPNE-Kras^{G12D} cells was analyzed following RREB-1 knockdown with lentivirally-expressed shRNA. Short hairpin RNAs (shRNAs) targeting RREB1 were purchased from Open Biosystems. These constructs consist of the following shRNA sequences cloned into the pLKO.1 lentiviral vector. The vector was modified from the original backbone to contain a Neomycin-resistance cassette.

shRNA1
Hairpin sequence for TRCN0000022199:
CCGGCCAGTATGTTTCAAGGAGTTTCTCGAGAAACTCCTTGAAACATACT
GGTTTTT
shRNA2
Hairpin sequence for TRCN0000022200:
CCGGCCAGGAAACGAAAGAGGAGAACTCGAGTTCTCCTCTTTCGTTTCCT
GGTTTTT

HPNE-Kras^{G12D} cells were infected with the anti-RREB-1 lentivirus-shRNA construct and selected in neomycin. As demonstrated by qPCR, the resulting cell populations showed decreased expression of RREB-1 as compared to controls (FIG. 3B). Inhibition of RREB-1 reversed the downregulation of both the miR-143/145 primary transcript and the mature miRNAs that occurs in HPNE-Kras^{G12D} cells (FIG. 3B,C). These data establish the existence of a Kras-RREB-1 signaling pathway which leads to repression of miR-143/145 expression.

### Example 4

### The RAF/MEK/MapK Pathway is Required for Kras-Mediated Repression of miR-143/145

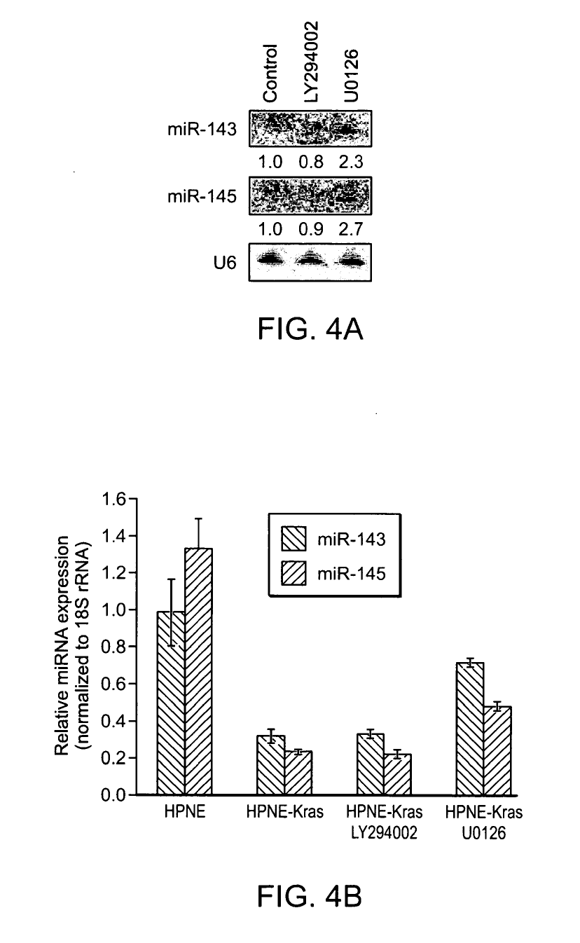

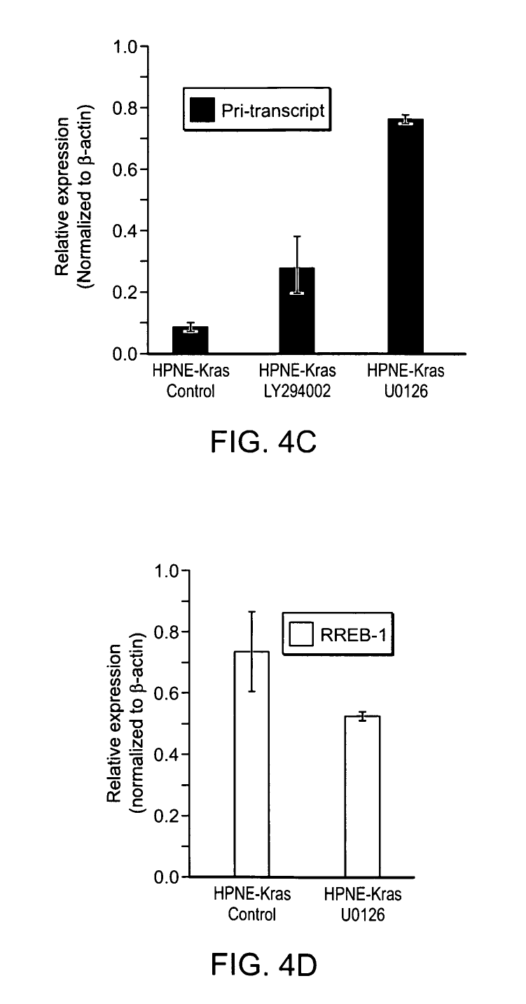

Kras activity leads to activation of both the Map Kinase (MAPK) cascade (also known as the RAF/MEK/MAPK pathway) and the phosphatidylinositol-3 kinase (PI3K) pathway. In order to determine which of these downstream effector pathways is involved in the Kras-RREB1-miR-143/145 pathway, levels of miR-143 and miR-145 were examined in HPNE-Kras^{G12D} cells treated with specific inhibitors that target MAPK and PI3K. Inhibition of MAPK by U0126 led to a significant increase of miR-143 and miR-145 expression as determined by northern blot and qRT-PCR, whereas the PI3K inhibitor LY294002 had no effect (FIG. 4A and FIG. 4B). Similarly, U0126 treatment, but not LY294002 treatment, increased the level of the miR-143/145 primary transcript (FIG. 4C). These results indicate that the RAF/MEK/MAPK pathway is necessary for the Kras-mediated repression of miR-143/145 in HPNE-Kras^{G12D} cells. Consistent with these results, RREB-1 expression as determined by qPCR was decreased in HPNE-Kras^{G12D} cells treated with MAPK inhibitor U0126 (FIG. 4D) suggesting that Ras, Mek, or some other member of the MAPK pathway is required to activate RREB-1 and thus maintain repression of miR-143/145. These results indicate that inhibition of the MAPK pathway with small molecules or other inhibitors represents one method to increase expression of miR-143/145 in the setting of activated Kras signaling. Since miR-143/145 have potent anti-tumorigenic effects (shown below), reactivation of expression of these miRNAs by MAPK-inhibition or other means represents a novel anti-cancer strategy.

### Example 5

### Expression of miR-143/145 in Pancreatic Tumor Cells Abrogated Tumor Formation

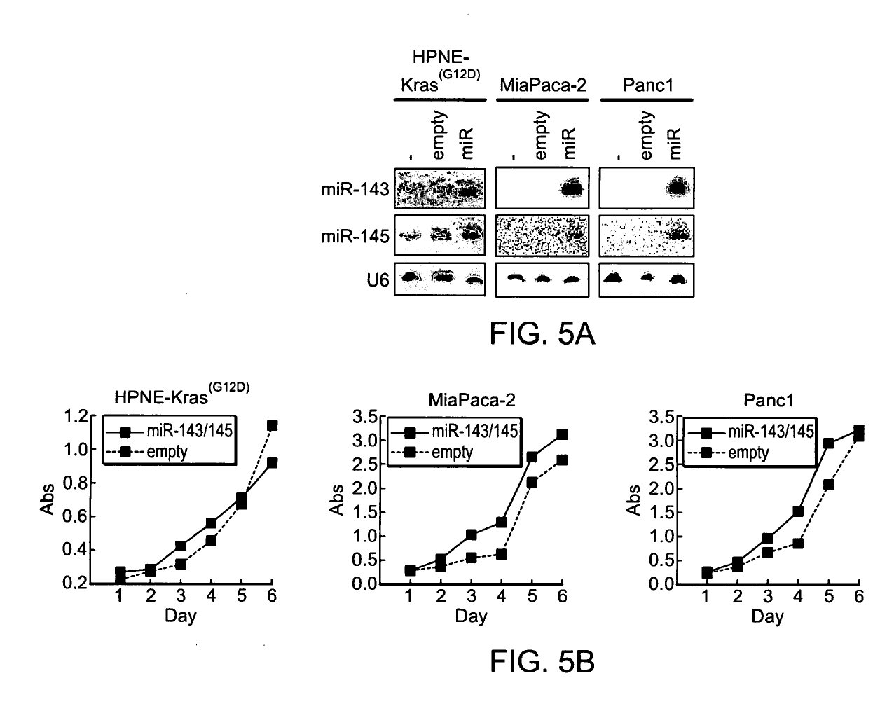

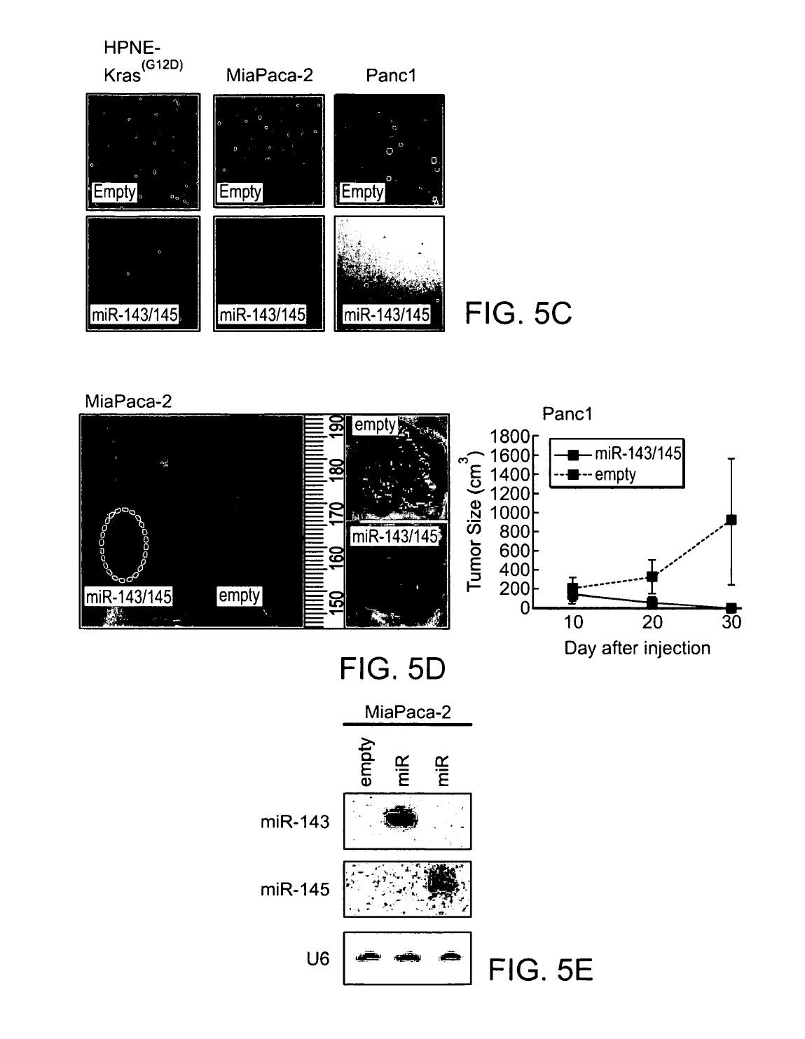

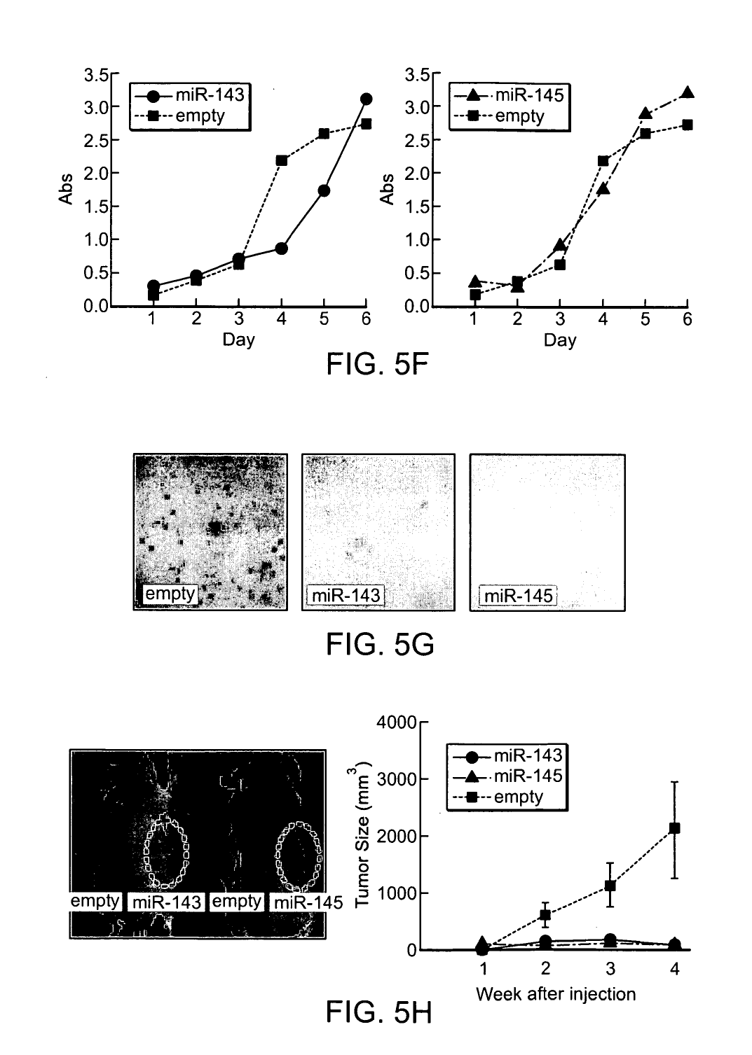

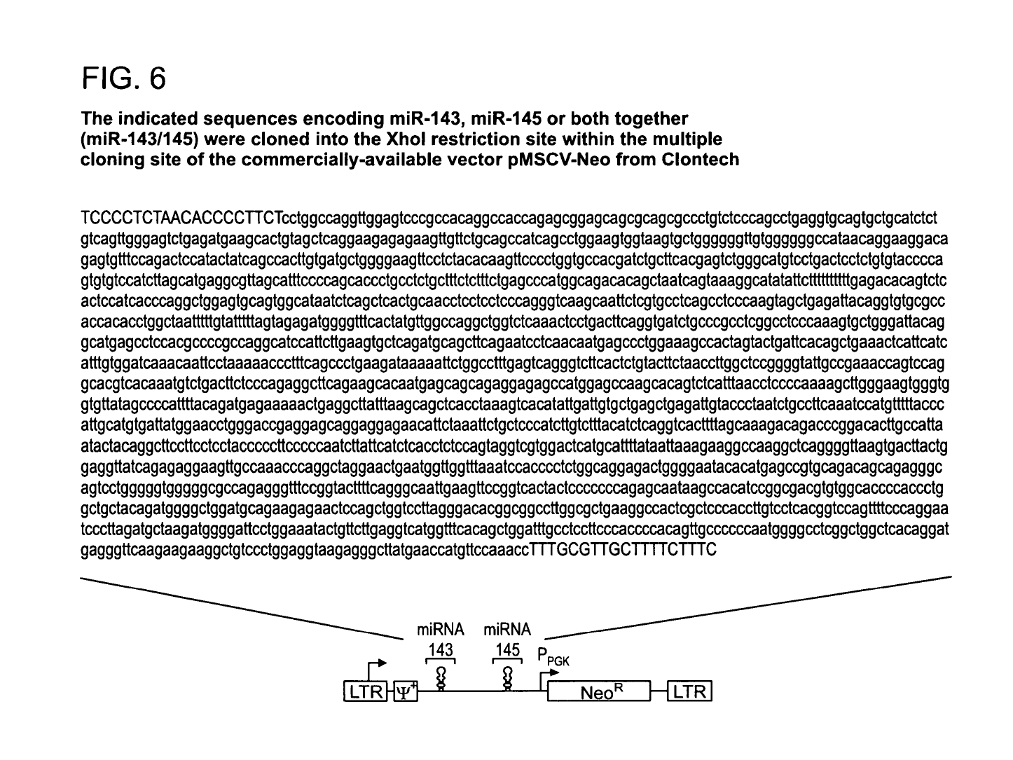

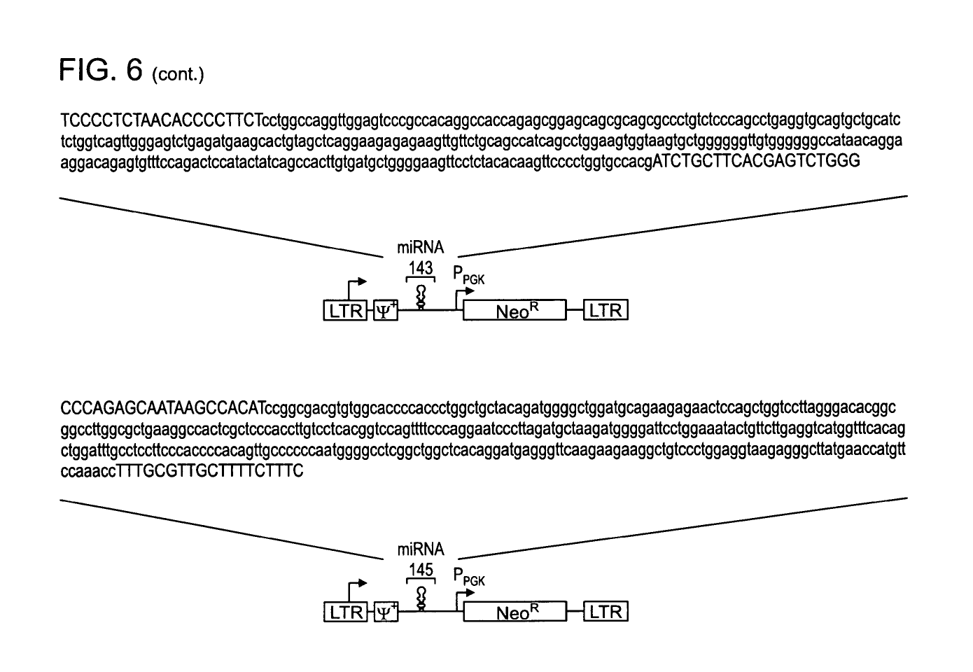

The functional consequences of Kras-mediated regulation of miR-143/145 was investigated by re-expressing these miRNAs in HPNE-Kras^{G12D} and two pancreatic cancer cell lines (MiaPaCa-2 and Panc1). Retroviral miRNA expression constructs were generated by amplifying and cloning segments of human genomic DNA encompassing the miR-143/145 cluster into a derivative of the murine stem cell virus (MSCV-Neo). The resulting construct expressed the miRNAs from the long terminal repeat (LTR) promoter and the neomycin resistance gene (NeoR) from an independent promoter. HPNE-Kras^{G12D}, MiaPaCa-2, and Panc1 were infected with this virus and selected in neomycin. Sequence information for these miRNA expression constructs is provided at FIG. 6. The resulting cell populations stably expressed the desired miRNAs as demonstrated by northern blotting (FIG. 5A).

Expression of miR-143/145 in HPNE-Kras^{G12D}, MiaPaCa-2, and Panc1 cell lines did not significantly influence the rate of proliferation in culture (FIG. 5B). In contrast, these miRNAs dramatically inhibited anchorage-independent growth of these cells (FIG. 5C). Furthermore, infection of MiaPaCa-2 or Panc-1 cells with the miR-143/145 virus completely abrogated tumor formation when these cells were injected into nude mice (FIG. 5d). MiaPaCa-2 and Panc-1 cells infected with empty virus were still able to produce tumors as expected. Thus, downregulation of miR-143/145 in tumor cells is absolutely required to maintain the tumorigenic phenotype. Therefore, restoration of miR-143/145 expression through delivery of synthetic miRNAs, delivery of plasmid or viral expression constructs that produce the miRNAs, or inhibition of the Kras-MAPK-RREB-1 signaling pathway all represent promising anti-cancer therapies.

In an attempt to attribute the observed phenotypic effects of miR-143/145 to either of the individual miRNAs, retroviral expression constructs were created similar to those described above that express either miR-143 or miR-145. MiaPaCa-2 cells were infected with these viruses and selected in neomycin. Northern blotting confirmed that the resulting cell populations stably expressed the desired miRNAs (FIG. 5E). As expected, expression of miR-143 or miR-145 individually in MiaPaCa-2 did not significantly influence the rate of proliferation in culture (FIG. 5F); however, individually these miRNAs both dramatically inhibited anchorage-independent growth of these cells (FIG. 5G). Likewise, infection of MiaPaCa-2 cells with the miR-143 or miR-145 virus completely abrogated tumor formation when these cells were injected into nude mice to a similar extent as expressing both miRNAs together (FIG. 5D and FIG. 5H). Thus, delivery or re-expression of miR-143 or miR-145 individually also represents a promising anti-cancer therapeutic strategy.

Together these results establish a signaling pathway in which Kras signaling, through the RAF/MEK/MAPK cascade, induces RREB-1 expression, resulting in repression of miR-143/145 expression. Given that re-expression of these miRNAs potently inhibited tumorigenesis, these data indicate that delivery or re-activation of miR-143, miR-145, and potentially other miRNAs that are repressed by Kras signaling such as miR-27b represents a novel therapeutic strategy for cancer. Similarly, inhibition of miRNAs that are induced by Kras, such as miR-31 and miR-199b, also is likely to provide an effective therapeutic strategy.

### REFERENCES

Bandres E., Cubedo E., Agirre X., Malumbres R., Zarate R., Ramirez N., Abajo A., Navarro A., Moreno I., Monzo M., and Garcia-Foncillas J. (2006). Identification by Real-time PCR of 13 mature microRNAs differentially expressed in colorectal cancer and non-tumoral tissues. Mol Cancer. 5.
Calin, G. A., et al., Human microRNA genes are frequently located at fragile sites and genomic regions involved in cancers. Proc Natl Acad Sci USA, 2004. 101(9): p. 2999-3004.
Calin, G. A., et al., A MicroRNA signature associated with prognosis and progression in chronic lymphocytic leukemia. N Engl J Med, 2005. 353(17): p. 1793-801.
Chang, T. C., et al., Transactivation of miR-34a by p53 Broadly Influences Gene Expression and Promotes Apoptosis. Mol Cell, 2007. 26(5): p. 745-52.
Chang, T. C., et al., Widespread microRNA repression by Myc contributes to tumorigenesis. Nat Genet. 40(1): p. 43-50.
Ciafre S. A., Galardi S., Mangiola A., Ferracin M., Liu C. G., Sabatino G., Negrini M., Maira G., Croce C. M., and Farace M. G. (2005). Extensive modulation of a set of microRNAs in primary glioblastoma. Biochem Biophys Res Commun. 334:1351-1358.
Date, S. et al. Finb, a multiple zinc finger protein, represses transcription of the human angiotensinogen gene. Int J Mol Med 13, 637-42 (2004).
He H., Jazdzewski K., Li W., Liyanarachchi S., Nagy R., Volinia S., et al. (2005a). The role of microRNA genes in papillary thyroid carcinoma. Proc. Natl. Acad. Sci. U.S.A. 102: 19075-19080.
He, L., et al., (2007). A microRNA component of the p53 tumour suppressor network. Nature. 447(7148):p. 1130-4.
Iorio M. V., Ferracin M., Liu C. G., Veronese A., Spizzo R., Sabbioni S., et al. (2005). MicroRNA gene expression deregulation in human breast cancer. Cancer Res 65: 7065-7070.
Jiang, J., Lee, E. J., Gusev, Y., and Schmittgen, T. D. (2005). Real-time expression profiling of microRNA precursors in human cancer cell lines. Nucleic Acids Res. 33: 5394-5403.
Kent O. A., and Mendell J. T. (2006). A small piece in the cancer puzzle: microRNAs as tumor suppressors and oncogenes. Oncogene 25: 6188-6196.
Metzler M., Wilda M., Busch K., Viehmann S., and Borkhardt A. (2003). High expression of precursor microRNA-155/BIC RNA in children with Burkitt lymphoma. Genes Chromosomes Cancer 37: 20-28.
Michael M. Z., S M O C, van Holst Pellekaan N. G., Young G. P., and James R. J. (2003). Reduced accumulation of specific microRNAs in colorectal neoplasia. Mol Cancer Res 1: 882-891.
Mukhopadhyay, N. K. et al. The zinc finger protein ras-responsive element binding protein-1 is a coregulator of the androgen receptor: implications for the role of the Ras pathway in enhancing androgenic signaling in prostate cancer. Mol Endocrinol 21, 2056-70 (2007).
Oxford, G., Smith, S. C., Hampton, G. & Theodorescu, D. Expression profiling of Ral-depleted bladder cancer cells identifies RREB-1 as a novel transcriptional Ral effector. Oncogene 26, 7143-52 (2007).
Pallante P., Visone R., Ferracin M., Ferraro A., Berlingieri M. T., Troncone G., Chiappetta G., Liu C. G., Santoro M., Negrini M., Croce C. M., and Fusco A. (2006). MicroRNA deregulation in human thyroid papillary carcinomas. Endocr Relat Cancer. 13: 497-508.
Sherr, C. J. and J. D. Weber (2000). The ARF/p53 pathway. Curr Opin Genet Dev. 10: p. 94-9.
Thiagalingam, A. et al. RREB1, a Ras Responsive Element Maps to Human Chromosome 6p25. Mol Cell Biol 16, 5335-45 (1996).
Thiagalingam, A. et al. RREB-1, a novel zinc finger protein, is involved in the differentiation response to Ras in human medullary thyroid carcinomas. Genomics 45, 630-632 (1997).
Uren, A. G. et al. Large-scale mutagenesis in p19(ARF)- and p53-deficient mice identifies cancer genes and their collaborative networks. Cell 133, 727-41 (2008).
Weber F, Teresi R E, Broelsch C E, Frilling A, and Eng C. J (2006). A limited set of human MicroRNA is deregulated in follicular thyroid carcinoma. Clin Endocrinol Metab. 91: 3584-3591.
Yanaihara N., Caplen N., Bowman E., Seike M., Kumamoto K., Yi M., et al. (2006). Unique microRNA molecular profiles in lung cancer diagnosis and prognosis. Cancer Cell 9: 189-198.
Zhang, S. et al. p16 INK4a gene promoter variation and differential binding of a repressor, the ras-responsive zinc-finger transcription factor, RREB. Oncogene 22, 2285-95 (2003).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of preventing or reducing tumorogenesis in a subject, the method comprising administering to the subject an agent that increases miR-143, miR-145, and/or miR-27b expression relative to a reference, thereby reducing or preventing tumor formation.

2. A method of treating or preventing a neoplasia in a subject, the method comprising administering to the subject an agent that increases miR-143, miR-145, and/or miR-27b expression relative to a reference, thereby treating or preventing the neoplasia.

3. A method of increasing miR-143, miR-145, and/or miR-27b expression in a neoplastic cell, the method comprising contacting the cell with an agent that increases miR-143, miR-145, and/or miR-27b expression relative to a reference.

4. (canceled)

5. A method of treating or preventing a neoplasia, the method comprising contacting a neoplastic cell having increased Kras signaling with an agent that inhibits a MAPK signaling pathway component or increases miR-143, miR-145, and/or miR-27b expression relative to a reference, thereby treating or preventing the neoplasia.

6. The method of claim 1, wherein the neoplasia is a pancreatic cancer.

7. The method of claim 1, wherein the untreated neoplastic cell is characterized as having a reduced level of miR-143, miR-145, and/or miR-27b.

8. The method of claim 1, wherein the agent inhibits the expression or biological activity of a component of a MAPK signaling pathway component.

9. The method of claim 8, wherein the agent is PD98059 [2′-amino-3′-methoxyflavone], PD184352 (CI-1040) [2-(2-chloro-4-iodo-phenylamino)-Ncyclopropylmethoxy-3,4-difluoro-benzamide] or U0126 [1,4-diamino-2,3-dicyano-1,4-bis(2-aminophenylthio)butadiene].

10. The method of claim 1, wherein the agent is an expression vector comprising a polynucleotide encoding miR-143, miR-145, and/or miR-27b.

11. An expression vector comprising a polynucleotide encoding miR-143, miR-145, and/or miR-27b positioned for expression in a mammalian cell, or
a vector comprising a polynucleotide encoding a miR-31, miR-34a and miR-199b inhibitory nucleic acid molecule.

12-15. (canceled)

16. A pharmaceutical composition for the treatment of neoplasia comprising an effective amount of an isolated miR-143, miR-145, and/or miR-27b polynucleotide in a pharmaceutically acceptable excipient, or
a pharmaceutical composition for the treatment of neoplasia comprising an effective amount of a vector encoding a miR-143, miR-145, and/or miR-27b microRNA in a pharmaceutically acceptable excipient, or
a kit for the treatment of a neoplasia, the kit comprising an effective amount of an agent that increases miR-143, miR-145, and/or miR-27b expression, and written instructions for using the kit, or
a pharmaceutical composition for the treatment of a neoplasia, the composition comprising an effective amount of a miR-31, miR-34a and miR-199b inhibitory nucleic acid molecule.

17-20. (canceled)

21. A method for identifying or characterizing a neoplasia in a subject, the method comprising detecting miR-143, miR-145, miR-27b, miR-31, miR-34a and miR-199b in a biological sample derived from the subject, thereby identifying or characterizing the neoplasia, or
a method for diagnosing a subject as having or having a propensity to develop a neoplasia, the method comprising
(a) measuring the level of a marker selected from the group consisting of miR-143, miR-145, miR-27b, miR-31, miR-34a and miR-199b in a biological sample from the subject, and
(b) detecting an alteration in the level of the marker in the sample relative to the level in a control sample, wherein detection of an alteration in the marker level indicates the subject has or has a propensity to develop a neoplasia, or
a method for identifying the prognosis of a subject having a neoplasia, the method comprising detecting the level of miR-143, miR-145, miR-27b level, miR-31, miR-34a and/or miR-199b in a subject, wherein an decrease in the level of miR-143, miR-145, miR-27b level or an increase in the level of miR-31 and miR-199b identifies the subject as having a poor prognosis.

22-25. (canceled)

26. A method for selecting a therapy for a subject having a neoplasia, the method comprising detecting Kras or MAPK signaling in a biological sample derived from the subject, wherein the level of Kras signaling or a increase in MAPK signaling relative to a reference is indicative of the efficacy of said therapy, or
a method for selecting a therapy for a subject having a neoplasia, the method comprising detecting miR-143, miR-145, miR-27b, miR-31, miR-34a and/or miR-199b in a biological sample derived from the subject, wherein the level of miR-143, miR-145, miR-27b, miR-31 and/or miR-199b relative to a reference is indicative of the efficacy of said therapy.

27-30. (canceled)

31. A microarray comprising at least two polynucleotides selected from the group consisting of miR-143, miR-145, miR-27b, miR-31, miR-34a and miR-199b, or
a nucleic acid probe that hybridizes with a microRNA sequence selected from the group consisting of miR-143, miR-145, miR-27b, miR-31, miR-34a and miR-199b

32-34. (canceled)

35. A method of preventing or reducing tumor formation, the method comprising contacting a neoplastic cell with an agent that reduces miR-31, miR-34a and/or miR-199b expression or biological activity, thereby reducing or preventing tumor formation, or
a method of treating or preventing a neoplasia in a subject, the method comprising administering to the subject an agent that reduces miR-31, miR-34a and/or miR-199b expression, thereby treating or preventing the neoplasia, or
a method for increasing miR143 and/or miR145 expression in a cell, the method comprising contacting the cell with an agent that inhibits RREB-1, thereby increasing the expression of miR-143 and/or 145 primary transcript or mature miRNA, or
a method for increasing miR143 and/or miR145 expression in a cell, the method comprising contacting the cell with an agent that inhibits a component of a MAPK pathway, thereby increasing the expression of miR-143 and/or 145.

37-47. (canceled)

48. A method of identifying an agent that treats or prevents a neoplasm, the method comprising
(a) contacting a cell that expresses a microRNA selected from the group consisting of miR-143, miR-145, miR-27b, miR-31, miR-34a and miR-199b with an agent, and
(b) comparing the level of microRNA expression in the cell contacted by the agent with the level of expression in a control cell, wherein an agent that alters microRNA expression thereby treats or prevents a neoplasm.

49-52. (canceled)


SEQUENCE LISTING (17 sequences)

SEQ ID NO: 1 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 2 - protein, 189 aa, Homo sapiens [sequence omitted]

SEQ ID NO: 3 - DNA, 945 bp, Homo sapiens
  modified_base (195)..(195): a, c, g, t, unknown or other
gacccctcccccgcaccgacgtgattcggatcgcgcggtgctggcgccgccttcatgcgccctgcctggcccccacctggtcctctttccttttcaggtggaggagatgccgctgtcccgtcggtctggggacagcccagctccccggatcccgggctggagagacgcgtcgcggccccggggcctggtggcacngagcaggaaggaggacccggcggcgggctctgcctgggcttgcctgggcttgttccgagccgggctgcttctcggtgaccacgcagatcgggggcatttggagattttgcgggagtcctgcagccaagctccggggcaggagaggcctggaagcctgcactacctgctcgccccgtcccagcatgcacccaggtgctggggagaggcaggacaggcctgtcccccgagtcccctccggatgccgtggaccggccagctgtgagtgtttctttggcagtgtcttagctggttgttgtgagcaatagtaaggaagcaatcagcaagtatactgccctagaagtgctgcacgttgtggggcccaagagggaagatgaagcgagagatgcccagaccagtgggagacgccaggacttcggaagctcttctgcgccacggtgggtggtgagggcggctgggaaagtgagctccagggccccaggagcagcctgctcgtgggtgcggaaggaaaaaggcacaggggcttggtgtgggcggcttttggctgggagaagtttgcacgtagggagaatagtagccagtgtttgcagagcacttactatgcaggaaggcctgtcctaagtattgtaagtgtattacatcatgtacaagtgtctgtgattaaccccgtcttgcagagaaggaaacaaaagtacaaacagaaaatgtaactaagcatgcaattaataaaaagggaccaggttttgaacgcga

SEQ ID NO: 4 - RNA, 22 nt, Homo sapiens
uggcagugucuuagcugguugu

SEQ ID NO: 5 - DNA, 23 bp, Homo sapiens
cccagtgtttagactatctgttc

SEQ ID NO: 6 - DNA, 21 bp, Homo sapiens
ggcaagatgctggcatagctg

SEQ ID NO: 7 - RNA, 21 nt, Homo sapiens
uucacaguggcuaaguucugc

SEQ ID NO: 8 - DNA, 21 bp, Homo sapiens
tgagatgaagcactgtagctc

SEQ ID NO: 9 - DNA, 21 bp, Homo sapiens
gtccagttttcccaggaatcc

SEQ ID NO: 10 - DNA, 57 bp, Artificial Sequence
  Description of Artificial Sequence Synthetic oligonucleotide
ccggccagtatgtttcaaggagtttctcgagaaactccttgaaacatactggttttt

SEQ ID NO: 11 - DNA, 57 bp, Artificial Sequence
  Description of Artificial Sequence Synthetic oligonucleotide
ccggccaggaaacgaaagaggagaactcgagttctcctctttcgtttcctggttttt

SEQ ID NO: 12 - DNA, 2973 bp, Artificial Sequence
  Description of Artificial Sequence Synthetic polynucleotide
agctcatataaggtaaaaggcagagtggcctctgtggccagggagccgcaggcaagggactaaggggaggggggctcagtgccagctgcttaaaaatgcccctgtggcagcgaggggcaccagaggctgggtctaattagttgagaagcagtgacacccccaaccactccccaaacaggctggctcccgtctccaggccccaaggagccacacctggaccagaccccaggaaagaagggcgctagctggtgggagccaccccgccatgctgatgtcagagaagcaagaatctggagaagcagcctcctgggaccagaggagggccagcagcaggcagcccggagacagaactctcacccacgcctctccaagcctcccaacagaagacagaggtcccccacagccagagacatttcctgaagacatggggaacacagaggcagaaacagcccatccacccaggagctgtcccccacactgccgggagccggcacccagagccgccaggtaaaactgaggccacctggttcaacatcacctttcacagaaggggaagcagccacagaaagaagggcctcgttaagaagtggaacctgggacccccaagcggtgtctctcatcctgactggggatccagagtaggagggagcctttggtgggacacaaaggaacagaacgaggcaccaacagcaaggggcaagcagattcaagggccacagaggagatggaggcaaacaccttcccctggtcagaggctgtgcctcagcccttctccctgcatcagtttctccttcagaagcatgggactacctcccatctagttctcgtttctaaacctaggggagatgctatctttgctgcaataatcttagcctacatcttggaatggaaatggccttggtggaaatggtcttcaactcctctggtccaagctcaggccctgtgaccctggaacaatccccttcctggtcctccatgtaggagcaataacattcccttgccagcagggttggagtcccgccacaggccaccagagcggagcagcgcagcgccctgtctcccagcctgaggtgcagtgctgcatctctggtcagttgggagtctgagatgaagcactgtagctcaggaagagagaagttgttctgcagccatcagcctggaagtggctggatgtgggaacttcagcactgccgtactcttgggaaatttgtccaaggccacccggctgagcagcggttgaaccaggacaccatcaggcatgcgtttcttgtctccaccacaccctcaacccacttcccaacgcgccttgcgacaggggctgcggtattgcatccacatgactgataaaaaatggaaaatttttcaaggtagagaagatgaaaactcagaacgccctcttgtggcatctctacccaccctaggaacactatggctcttcccctacacatggtgattgctaaccttgctacaagacgttggacacacacacacacacacacacacacacacacacactgaggttccttttgccccctcacttttgagccagtgactactgaaaccctctccattgttgcaccaccagcaatgcccccatcacttcctctcatttacttccacaggctggttcatcctcaaagccctccttacgtagatctgtgggatcagtgaggctcagagaggtaaagtggccagcccaaggtcgcccagacagcaaaaggcagggccagcgctgatttcaagtccaatggcctatggcaatttcttagccaaaagcaaaatctacaaaaataaaaagtcaggcacagtggtgagtgcctacagtcccagctactgaggaggccgacgggggaggaccacttgagcttgggagttcctggctgcagagagctatgattgtgcctgtgaatagccaatgcactccagcctgagcaagatagggagaccctgtctctaaaaaatacctaaataattttaaaagtcagcctctctgactgcctatagagaatgctaactaactgaatgacagaagacctaatgtaatccaggtgcaaaatcagaactttccggccgggcgcggtggctcacacctgtaatcccaacactttgggaggcccaggcgggtggatcacgaggtcaggagttcaagaccggcctgcccaacatggcaaaaccccgtctctactaaaaatacaaaaaattagctgggcatggtggtggccacctataatctcagctactcaggaggctgaggcaggagaattgcttggacccgggaggcagaggttgcagtgagctgagatcgcgccactgcactccagcgtgggggacaaaagcgaaactctgtctcaaaaaaaaaaaaattagaactttccccgtactcttgctagggcttttcatggagatgtagaaatggtagtaagtgccaaggccccagaaccctcatgtttgggtccgactcccacattgccagagactaggcagctcacacaggtgtcccaagctgtctttctcacaggccgcattgaaggcatttatgaaatgagaccccctcttcctcatccgtagtgacagggctggcctgaccgtggagaaatcagatttgatcatgatagcttccactccgtgactggtcacgatctgccaggcgctgtgctaaacactttcataggccttgtccccctccatcctgataacaccccagtgaagcaggccctgctcttcactccactccacaaagccaaggctgagagaggttaacagacttacctaaggtcacacagctaagaagtcgtgaagctgggatcgaatcaagtctgtgtttatgggtttaactatcatgttgtggccccaaccccatttgacctagcctggctggtgggccttcttgcagtagcttcccctggagaagaggaaaagcaaaccttcattgagacccaagcggtctctcctgtgctctgtgacaataataaa

SEQ ID NO: 13 - DNA, 14 bp, Homo sapiens
ccccaaccactccc

SEQ ID NO: 14 - DNA, 14 bp, Artificial Sequence
  Description of Artificial Sequence Synthetic consensus oligonucleotide
ccccaaaccacccc

SEQ ID NO: 15 - DNA, 2030 bp, Artificial Sequence
  Description of Artificial Sequence Synthetic polynucleotide
tcccctctaacaccccttctcctggccaggttggagtcccgccacaggccaccagagcggagcagcgcagcgccctgtctcccagcctgaggtgcagtgctgcatctctggtcagttgggagtctgagatgaagcactgtagctcaggaagagagaagttgttctgcagccatcagcctggaagtggtaagtgctggggggttgtggggggccataacaggaaggacagagtgtttccagactccatactatcagccacttgtgatgctggggaagttcctctacacaagttcccctggtgccacgatctgcttcacgagtctgggcatgtcctgactcctctgtgtaccccagtgtgtccatcttagcatgaggcgttagcatttccccagcaccctgcctctgctttctctttctgagcccatggcagacacagctaatcagtaaaggcatatattcttttttttttgagacacagtctcactccatcacccaggctggagtgcagtggcataatctcagctcactgcaacctcctcctcccagggtcaagcaattctcgtgcctcagcctcccaagtagctgagattacaggtgtgcgccaccacacctggctaatttttgtatttttagtagagatggggtttcactatgttggccaggctggtctcaaactcctgacttcaggtgatctgcccgcctcggcctcccaaagtgctgggattacaggcatgagcctccacgccccgccaggcatccattcttgaagtgctcagatgcagcttcagaatcctcaacaatgagccctggaaagccactagtactgattcacagctgaaactcattcatcatttgtggatcaaacaattcctaaaaaccctttcagccctgaagataaaaattctggcctttgagtcagggtcttcactctgtacttctaaccttggctccggggtattgccgaaaccagtccaggcacgtcacaaatgtctgacttctcccagaggcttcagaagcacaatgagcagcagaggagagccatggagccaagcacagtctcatttaacctccccaaaagcttgggaagtgggtggtgttatagccccattttacagatgagaaaaactgaggcttatttaagcagctcacctaaagtcacatattgattgtgctgagctgagattgtaccctaatctgccttcaaatccatgtttttacccattgcatgtgattatggaacctgggaccgaggagcaggaggagaacattctaaattctgctcccatcttgtctttacatctcaggtcacttttagcaaagacagacccggacacttgccattaatactacaggcttccttcctcctacccccttcccccaatcttattcatctcacctctccagtaggtcgtggactcatgcattttataattaaagaaggccaaggctcaggggttaagtgacttactggaggttatcagagaggaagttgccaaacccaggctaggaactgaatggttggtttaaatccacccctctggcaggagactggggaatacacatgagccgtgcagacagcagagggcagtcctgggggtgggggcgccagagggtttccggtacttttcagggcaattgaagttccggtcactactcccccccagagcaataagccacatccggcgacgtgtggcaccccaccctggctgctacagatggggctggatgcagaagagaactccagctggtccttagggacacggcggccttggcgctgaaggccactcgctcccaccttgtcctcacggtccagttttcccaggaatcccttagatgctaagatggggattcctggaaatactgttcttgaggtcatggtttcacagctggatttgcctccttcccaccccacagttgccccccaatggggcctcggctggctcacaggatgagggttcaagaagaaggctgtccctggaggtaagagggcttatgaaccatgttccaaacctttgcgttgcttttctttc

SEQ ID NO: 16 - DNA, 326 bp, Homo sapiens
tcccctctaacaccccttctcctggccaggttggagtcccgccacaggccaccagagcggagcagcgcagcgccctgtctcccagcctgaggtgcagtgctgcatctctggtcagttgggagtctgagatgaagcactgtagctcaggaagagagaagttgttctgcagccatcagcctggaagtggtaagtgctggggggttgtggggggccataacaggaaggacagagtgtttccagactccatactatcagccacttgtgatgctggggaagttcctctacacaagttcccctggtgccacgatctgcttcacgagtctggg

SEQ ID NO: 17 - DNA, 371 bp, Homo sapiens
cccagagcaataagccacatccggcgacgtgtggcaccccaccctggctgctacagatggggctggatgcagaagagaactccagctggtccttagggacacggcggccttggcgctgaaggccactcgctcccaccttgtcctcacggtccagttttcccaggaatcccttagatgctaagatggggattcctggaaatactgttcttgaggtcatggtttcacagctggatttgcctccttcccaccccacagttgccccccaatggggcctcggctggctcacaggatgagggttcaagaagaaggctgtccctggaggtaagagggcttatgaaccatgttccaaacctttgcgttgcttttctttc